SEQUENCE LISTING (35 sequences)

SEQ ID NO: 1 - protein, 146 aa, Homo sapiens
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - protein, 5 aa, Artificial Sequence
  n-mer that can be inserted in SEQ ID NO. 1 (to create an experimentally altered version of SEQ ID NO. 1)
RVQFQ

SEQ ID NO: 3 - protein, 5 aa, Artificial Sequence
  n-mer that can be inserted in SEQ ID NO. 1 (to create an experimentally altered version of SEQ ID NO. 1)
CVEIP

SEQ ID NO: 4 - protein, 7 aa, Artificial Sequence
  n-mer that can be inserted in SEQ ID NO. 1 (to create an experimentally altered version of SEQ ID NO. 1)
GSGSGSC

SEQ ID NO: 5 - protein, 8 aa, Artificial Sequence
  n-mer that can be inserted in SEQ ID NO. 1 (to create an experimentally altered version of SEQ ID NO. 1)
IEALTPHS

SEQ ID NO: 6 - protein, 8 aa, Artificial Sequence
  n-mer that can be inserted in SEQ ID NO. 1 (to create an experimentally altered version of SEQ ID NO. 1)
YGQRQWKN

SEQ ID NO: 7 - protein, 9 aa, Artificial Sequence
  n-mer that can be inserted in SEQ ID NO. 1 (to create an experimentally altered version of SEQ ID NO. 1)
VFIINNSLE

SEQ ID NO: 8 - protein, 44 aa, Artificial Sequence
  n-mer that can be inserted in SEQ ID NO. 1 (to create an experimentally altered version of SEQ ID NO. 1)
RAASAGSYSEWSMTPRFTPWWETKIDPPVMNITQVNGSLLVILH

SEQ ID NO: 9 - protein, 5 aa, Artificial Sequence
  n-mer that can be added as an N-terminal peptide on SEQ ID NO. 1 (to create an experimentally altered version of SEQ ID NO. 1)
AEPEE

SEQ ID NO: 10 - protein, 6 aa, Artificial Sequence
  n-mer that can be added as an N-terminal peptide on SEQ ID NO. 1 (to create an experimentally altered version of SEQ ID NO. 1)
ACEPEE

SEQ ID NO: 11 - protein, 8 aa, Artificial Sequence
  n-mer that can be added as an N-terminal peptide on SEQ ID NO. 1 (to create an experimentally altered version of SEQ ID NO. 1)
GPACEPEE

SEQ ID NO: 12 - protein, 13 aa, Artificial Sequence
  n-mer that can be added as an N-terminal peptide on SEQ ID NO. 1 (to create an experimentally altered version of SEQ ID NO. 1)
GGSSGSGSEVLFQ

SEQ ID NO: 13 - protein, 13 aa, Artificial Sequence
  n-mer that can be added as an N-terminal peptide on SEQ ID NO. 1 (to create an experimentally altered version of SEQ ID NO. 1)
GSGSGSCGSGSGS

SEQ ID NO: 14 - protein, 14 aa, Artificial Sequence
  n-mer that can be added as an N-terminal peptide on SEQ ID NO. 1 (to create an experimentally altered version of SEQ ID NO. 1)
GGSSGSGSEVLFQG

SEQ ID NO: 15 - protein, 15 aa, Artificial Sequence
  n-mer that can be added as an N-terminal peptide on SEQ ID NO. 1 (to create an experimentally altered version of SEQ ID NO. 1)
GPGSGSGSCGSGSGS

SEQ ID NO: 16 - protein, 16 aa, Artificial Sequence
  n-mer that can be added as an N-terminal peptide on SEQ ID NO. 1 (to create an experimentally altered version of SEQ ID NO. 1)
GGSSGSGSEVLFQGPG

SEQ ID NO: 17 - protein, 21 aa, Artificial Sequence
  n-mer that can be added as an N-terminal peptide on SEQ ID NO. 1 (to create an experimentally altered version of SEQ ID NO. 1)
GGSSGSGSEVLFQGPACEPEE

SEQ ID NO: 18 - protein, 28 aa, Artificial Sequence
  n-mer that can be added as an N-terminal peptide on SEQ ID NO. 1 (to create an experimentally altered version of SEQ ID NO. 1)
GGSSGSGSEVLFQGPGSGSGSCGSGSGS

SEQ ID NO: 19 - protein, 7 aa, Artificial Sequence
  n-mer that can be added as a C-terminal peptide on SEQ ID NO. 1 (to create an experimentally altered version of SEQ ID NO. 1)
GSGSGSC

SEQ ID NO: 20 - protein, 149 aa, Artificial Sequence
  Experimentally altered version of SEQ ID NO. 1
GPGCPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 21 - protein, 161 aa, Artificial Sequence
  Experimentally altered version of SEQ ID NO. 1
GPGSGSGSCGSGSGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 22 - protein, 149 aa, Artificial Sequence
  Experimentally altered version of SEQ ID NO. 1
GPGCPISSHCRLDKSNFQQPYITNRTFMLAKEASLADQNTDVRLIGEKLFHGVSMSERCYLMKQVLQFTLEEVLFPQSDRFQPYMQEVVPFLARLSNRLSTCHIEGDDLHIQRNVQKLKDTVKKLGESGEIKAIGELDLLFMSLRNACI

SEQ ID NO: 23 - protein, 146 aa, Artificial Sequence
  Experimentally altered version of SEQ ID NO. 1
CPISSHCRLDKSNFQQPYITNRTFMLAKEASLADQNTDVRLIGEKLFHGVSMSERCYLMKQVLQFTLEEVLFPQSDRFQPYMQEVVPFLARLSNRLSTCHIEGDDLHIQRNVQKLKDTVKKLGESGEIKAIGELDLLFMSLRNACI

SEQ ID NO: 24 - protein, 147 aa, Artificial Sequence
  Experimentally altered version of SEQ ID NO. 1
GAPISSCCRLDKSNFQQPYITNRTFMLAKEASLADQNTDVRLIGEKLFHGVSMSERCYLMKQVLQFTLEEVLFPQSDRFQPYMQEVVPFLARLSNRLSTCHIEGDDLHIQRNVQKLKDTVKKLGESGEIKAIGELDLLFMSLRNACI

SEQ ID NO: 25 - protein, 146 aa, Artificial Sequence
  Experimentally altered version of SEQ ID NO. 1
APISSHCRLDKSNFQQPYITNRTFMLAKEASLCDQNTDVRLIGEKLFHGVSMSERCYLMKQVLQFTLEEVLFPQSDRFQPYMQEVVPFLARLSNRLSTCHIEGDDLHIQRNVQKLKDTVKKLGESGEIKAIGELDLLFMSLRNACI

SEQ ID NO: 26 - protein, 19 aa, Artificial Sequence
  His-tag
HHHHHHGGSSGSGSEVLFQ

SEQ ID NO: 27 - protein, 7 aa, Artificial Sequence
  Consensus sequence for native cut site in protease-cleavable TEV linker
ENLYFQS

SEQ ID NO: 28 - protein, 146 aa, Artificial Sequence
  Experimentally altered version of SEQ ID NO. 1
APISSHCRLDKSNFQQPYITNRTFMLAKEASLADQNTDVRLIGEKLFHGVSMSERCYLMKQVLQFTLEEVLFPQSDRFQPYMQEVVPFLARLSNRLSTCHIEGDDCHIQRNVQKLKDTVKKLGESGEIKAIGELDLLFMSLRNACI

SEQ ID NO: 29 - protein, 146 aa, Artificial Sequence
  Experimentally altered version of SEQ ID NO. 1
APISSHCRLDKSNFQQPYITNRTFMLAKEASLADQNTDVRLIGEKLFHGVSMSERCYLMKQVLQFTLEEVLFPQSDRFQPYMQEVVPFLARLSNCLSTCHIEGDDLHIQRNVQKLKDTVKKLGESGEIKAIGELDLLFMSLRNACI

SEQ ID NO: 30 - protein, 146 aa, Artificial Sequence
  Experimentally altered version of SEQ ID NO. 1
GPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 31 - protein, 146 aa, Artificial Sequence
  Experimentally altered version of SEQ ID NO. 1
GPISSHCRLDKSNFQQPYITDRTFMLAKEASLADDNTDVRLIGEKLFHGVSMSERCYLMKQVLDFTLEEVLFPQSDRFQPYMQEVVPFLARLSNRLSTCHIEGDDLHIQRNVQKLKDTVKKLGESGEIKAIGELDLLFMSLRNACI

SEQ ID NO: 32 - protein, 146 aa, Artificial Sequence
  Experimentally altered version of SEQ ID NO. 1
GPISSHCRLDKSNFQQPYITNRTFMLAKEASLADQNTDVRLIGEKLFHGVSMSERCYLMKQVLQFTLEEVLFPQSDRFQPYMQEVVPFLARLSNRLSTCHIEGDDLHIQRNVQKLKDTVKKLGESGEIKAIGELDLLFMSLRNACI

SEQ ID NO: 33 - protein, 146 aa, Artificial Sequence
  Experimentally altered version of SEQ ID NO. 1
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDCHIQRNVQKLKDTVKKLGESGEIKAIGELDLLFMSLRNACI

SEQ ID NO: 34 - protein, 146 aa, Artificial Sequence
  Experimentally altered version of SEQ ID NO. 1
CPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 35 - protein, 146 aa, Artificial Sequence
  Experimentally altered version of SEQ ID NO. 1
APISSHCRLDKSNFQQPYITNRTFMLAKEASLADNNTDVRLIGEKLFHGVSMSERCYLMKQVLNFTLEEVLFPQSDRFQPYMQEVVPFLARLSNCLSTCHIEGDDLHIQRNVQKLKDTVKKLGESGEIKAIGELDLLFMSLRNACI

(19) United States
(12) Patent Application Publication
(10) Pub. No.: US 2024/0239855 A1
(43) Pub. Date: Jul. 18, 2024

(54) THERAPEUTIC DERIVATIVES OF INTERLEUKIN-22

(71) Applicant: Cytoki Pharma ApS, Søborg (DK)

(72) Inventors: Kristian SASS-ØRUM, Bagsvæd (DK); Rasmus JØRGENSEN, Hellerup (DK); Sebastian Beck JØRGENSEN, Bagsvæd (DK); Henning THØGERSEN, Bagsvæd (DK); Thomas HOEG-JENSEN, Bagsvæd (DK); Michael Paolo Bastner SANDRINI, Bagsvæd (DK)

(21) Appl. No.: 18/560,085

(22) PCT Filed: May 11, 2022
(86) PCT No.: PCT/EP2022/062828

§ 371 Date: Nov. 9, 2023

(30) Foreign Application Priority Data
May 11, 2021 (EP) 21173310.0
May 11, 2021 (EP) 21173388.6

(51) Int. Cl. C07K 14/54; A61K 38/00; A61K 47/54; A61P 3/10

(52) U.S. Cl. CPC C07K 14/54; A61K 47/542; A61P 3/10; A61K 38/00

(57) ABSTRACT

The invention relates to novel derivatives of Interleukin-22 (IL-22), particularly those comprising a fatty monoacid covalently attached to an IL-22 protein, and their use in therapy.

### FIELD OF THE INVENTION

The present invention relates to novel derivatives of Interleukin-22 (IL-22), and in particular to derivatives comprising a fatty acid covalently attached to an IL-22 protein, wherein the fatty acid is a monoacid. The invention also encompasses methods for their production and their use in therapy, including the treatment, prevention and amelioration of metabolic, liver, pulmonary, gut, kidney, central nervous system (CNS) and skin diseases, disorders and conditions.

### BACKGROUND OF THE INVENTION

IL-22 is a 146 amino acid protein with a molecular weight of 17 KDa. It belongs to the IL-10 family of cytokines and selectively activates a heterodimeric receptor consisting of an IL-10 receptor B subunit (IL-10RA2), which is ubiquitously expressed, and an IL-22 receptor A subunit (IL-22RA1), which has an epithelial restricted expression. It is a unique cytokine in that it is released from immune cells, but selectively targets epithelial cells. Hence, the signaling pathways induced by IL-22 may have relevance in different tissues (targets include skin, intestine, lung, liver, kidney, pancreas and thymus), but IL-22 activates them in an epithelial-specific manner. A soluble binding protein, IL-22BP, neutralises IL-22 and thus regulates its effect.

IL-22 is released as a response to signals reflecting chemical or mechanical injury, e.g. aryl hydrocarbon receptor activation in response to environmental toxins or tryptophan intermediates, and the activation of pattern recognition receptors, such as toll-like receptor 4, in response to proteins, fragments and debris from dying cells or invading pathogens. IL-22 release is further stimulated by certain cytokines, in particular IL-23 and to a lesser extent IL1β. IL-22 is thus secreted as a response to cues reflecting pathogen infection and immune activation too.

The effect of IL-22 is the result of an orchestrated engagement of several activities/pathways. IL-22 acts on epithelial barrier tissues and organs upon injury to protect the cells and maintain barrier function (e.g. through activation of anti-apoptotic gene programs). It also accelerates repair (e.g. by inducing the proliferation of mature cells and activation of stem cells), prevents fibrosis (e.g. through reducing epithelial-mesenchymal transition, antagonising the NLRP3 inflammasome and inducing hepatic stellate cell senescence) and controls inflammation (e.g. by inducing anti-microbial peptides and chemotaxis signals). IL-22 has been reported as able to treat a range of medical conditions, including those often observed in diabetic or overweight mammals, such as hyperglycemia, hyperlipidemia and hyperinsulinemia.

However, IL-22 is generally cleared quickly from the body by the kidneys, which limits its use in clinical practice. This is a common feature of cytokines, and half-life extended cytokine drug development candidates have reached the drug development stage for treatment of e.g. oncology and immunotherapy. Generally these half-life extended cytokines use Fc fusion solutions or PEGylations. Known methods for extending the half-life of circulating IL-22 therefore seek to artificially increase the size of IL-22 beyond 70 kDa, so as to avoid renal clearance. Ligating IL-22 to an Fc antibody fragment is currently the best solution to this effect; Genentech and Generon Shanghai both have long-acting IL-22-Fc fusions in clinical development. Modifying IL-22 with polyethylene glycol (PEGylation) is another known means for avoiding renal clearance.

However, these existing solutions are not without their disadvantages. The available data suggest that PEG itself is immunogenic and PEG-containing vacuoles are observed in cells with PEGylated biologicals. Decreased activity and heterogeneity are also disadvantageous aspects of PEGylation. Although Fc fusion technology is very well known, adding an Fc antibody fragment represents a major change in the structure of IL-22, which affects its properties beyond half-life extension. As Fc fusion increases the size of the protein from approximately 17 kDa to approximately 85 kDa, properties such as diffusion rate, distribution and receptor engagement kinetics may be affected. For example, some Fc fusions are slowly absorbed and/or are too large for administration via certain routes. Both Genentech and Generon also report moderate and reversible skin reactions as dose-limiting adverse effects of IL-22-Fc fusions. Furthermore, the potency may be affected through steric hindrance caused by the large fusion partner.

In addition, while the native IL-22 has a very short half-life of a few hours, which would strongly limit its clinical utility, the IL-22-Fc fusions have been reported to have a half-life of a week or longer in man. For some conditions it will be beneficial to have potent molecules with prolonged half-life compared to native IL-22, but a shorter half-life than the Fc-fusions, e.g. for optimal titration, local application and action. There are currently no solutions described for this problem.

A need therefore remains in the art for new biocompatible modifiers of IL-22 that enhance circulating half-lives, and half-life in local tissue compartments and biological fluids, and demonstrate optimised pharmacokinetic and pharmacodynamic properties compared to the native molecule. Ideally, they should maintain potency and other properties of the native molecule and also avoid toxicity, immunogenicity and any other adverse reactions demonstrated by known derivatives.

### SUMMARY OF THE INVENTION

In a first aspect, there is provided a derivative of IL-22 comprising a fatty acid covalently attached to an IL-22 protein, wherein the fatty acid is a monoacid.

In embodiments of the invention, the fatty monoacid is covalently attached to the IL-22 protein by a linker.

The fatty monoacid may be of Formula I:

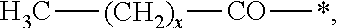

wherein x is an integer in the range of 10-18, optionally 12-18, 14-16 or 16-18, and * designates a point of attachment to the IL-22 protein or linker. It may be a C12, C14, C16, C18 or C20 monoacid. Advantageously, the fatty acid is a C16 or C18 monoacid, and most advantageously it is a C16 monoacid.

The IL-22 protein may be native mature human IL-22 (hereinafter “hIL-22”) or a variant thereof. The variant may be a substituted form of hIL-22, optionally substituted at position 1, 21, 35, 64, 113 and/or 114. It may comprise a substitution of hIL-22 selected from the group consisting of A1C, A1G, A1H, N21C, N21D, N21Q, N35C, N35D, N35H, N35Q, N64C, N64D, N64Q, N64W, Q113C, Q113R, K114C and K114R. Advantageously, the variant comprises a Cys residue at position 1 of hIL-22. It may comprise a Cys residue at position 95 or 106 of hIL-22. It may comprise a variation within hIL-22 and have at least 10% sequence identity with hIL-22. It may comprise one, two, three, four, five or more variations within hIL-22, wherein said variations are independently selected from the group consisting of deletions, substitutions and insertions.

The variant may be an extended form of hIL-22. It may comprise an N-terminal peptide, such as an N-terminal trimer. Advantageously, the variant comprises an N-terminal G-P-G.

It may comprise an N-terminal peptide of up to five, 10, 15, 20, 25, 30, 35, 40, 45 or 50 amino acids.

The linker may comprise one or more amino acids, optionally including glutamic acid (Glu) and/or lysine (Lys). The linker may include an oxyethylene glycine unit or multiple linked oxyethylene glycine units, optionally 2-5 such units, advantageously 2 units. The linker may comprise one or more oligo(ethylene glycol) (OEG) residues. It may comprise an ethylenediamine (C2DA) group and/or an acetamide (Ac) group. Advantageously, the linker comprises all of the aforementioned elements in combination. In particular, the linker may be γGlu-OEG-OEG-C2DA-Ac, γGlu-γGlu-γGlu-γGlu-OEG-OEG-εLys-αAc or γGlu-OEG-OEG-εLys-αAc.

The linker may additionally or alternatively be a Cys-reactive linker attached to a Cys residue in the hIL-22 or variant thereof. It may be attached at position −7, −5, 1, 6, 33, 113, 114 or 153 of the hIL-22 or variant thereof (where positions −7, −5 etc. are as defined herein). As an example, the linker may be attached to a Cys residue substituted at position 1, 6, 33, 113 or 114 of hIL-22. It may be attached to a Cys residue at position −5, −7 or 153 relative to hIL-22. Advantageously, the linker is attached to a Cys residue substituted at position 1 of hIL-22. In an embodiment, the linker is attached to a Cys residue substituted at position 95 or 106 of hIL-22.

In an embodiment, the derivative comprises a C14, C16, C18 or C20 monoacid covalently attached by a linker to a variant of hIL-22, wherein the variant optionally comprises an N-terminal G-P-G and a Cys residue at position 1 of hIL-22 and the linker is optionally attached to said Cys residue. An exemplary derivative of the invention is identified herein as Derivative 1. Another exemplary derivative of the invention is identified herein as Derivative 2.

In a second aspect, there is provided a process for preparing a derivative of the first aspect comprising covalently attaching a fatty monoacid to an IL-22 protein.

In a third aspect, there is provided a pharmaceutical composition comprising a derivative of the first aspect, and a pharmaceutically acceptable vehicle. The pharmaceutical composition is suitable for administration by inhalation, by injection, topically, orally or ocularly, optionally wherein the injection is intraperitoneal, subcutaneous or intravenous.

In a fourth aspect, there is provided a derivative of the first aspect or a pharmaceutical composition of the third aspect, for use in therapy.

In a fifth aspect, there is provided a derivative of the first aspect or a pharmaceutical composition of the second aspect, for use in a method of therapy, said method comprising administering a daily dose of between 0.001 μg/kg of body weight and 10 mg/kg of body weight of the derivative.

In a sixth aspect, there is provided a derivative of the first aspect or a pharmaceutical composition of the third aspect, for use in a method of treating a metabolic, liver, pulmonary, gut, kidney, CNS or skin disease, disorder or condition.

The metabolic disease, disorder or condition may be obesity, diabetes type 1, diabetes type 2, hyperlipidemia, hyperglycemia or hyperinsulinemia.

The liver disease, disorder or condition may be non-alcoholic fatty liver disease (NAFLD), non-alcoholic steatohepatitis (NASH), cirrhosis, alcoholic hepatitis, acute liver failure, chronic liver failure, acute-on-chronic liver failure (ACLF), acute liver injury, acetaminophen-induced liver toxicity, sclerosing cholangitis, biliary cirrhosis or a pathological condition caused by surgery or transplantation.

The pulmonary disease, disorder or condition may be chronic obstructive pulmonary disease (COPD), cystic fibrosis, bronchiectasis, idiopathic pulmonary fibrosis, acute respiratory distress syndrome, a chemical injury, a viral infection, a bacterial infection or a fungal infection.

The gut disease, disorder or condition may be inflammatory bowel disease (IBD), ulcerative colitis, Crohn's disease, graft-versus-host-disease (GvHD), a chemical injury, a viral infection or a bacterial infection.

The kidney disease, disorder or condition may be acute kidney disease or chronic kidney disease.

The CNS disease, disorder or condition may be multiple sclerosis.

The skin disease, disorder or condition may be a wound, inflammatory disease or GvHD.

### BRIEF DESCRIPTION OF THE DRAWINGS

FIG. 1 illustrates the structure of two derivatives of the invention identified herein as Derivative 1 (FIG. 1A) and Derivative 2 (FIG. 1B), respectively.

FIG. 2 illustrates the structure of a comparator for the derivatives of the invention identified herein as Comparator 1.

FIG. 3 illustrates the structure of a comparator for the derivatives of the invention identified herein as Comparator 2.

FIG. 4 illustrates examples of a (A) C18 diacid, (B) C16 diacid, and (C) C14 diacid, each connected to a linker comprising a Cys-reactive unit. These combinations of fatty acids and linkers are employed in the comparators for the derivatives of the invention identified herein as Comparators 4-13.

FIG. 5 illustrates the structure of an IL-22 protein identified herein as Comparator 4.

FIG. 6 illustrates the structure of an IL-22 protein herein as Comparator 9.

FIG. 7 illustrates the structure of an IL-22 protein identified herein as Comparator 13.

FIG. 8 illustrates the effect of daily dosing of hIL-22 and a comparative IL-22 variant having backbone variations only (identified herein as Comparator 16) on blood glucose in an 8-day study in a diabetes mouse model (mean±SEM).

FIG. 9 illustrates the effect of daily dosing of a comparator for the derivatives of the invention (herein identified as Comparator 4) compared to an IL-22-Fc fusion (specifically a human Fc N-terminally fused to hIL-22; hereinafter “hFc-hIL-22”) on (A) blood glucose, and (B) food intake, in a 16-day study in a diabetes mouse model (mean±SEM; * means p<0.05 using an unpaired t-test).

FIGS. 10A-C illustrate the effect of daily dosing of Comparator 4 and hFc-hIL-22 on three different target engagement biomarkers, in a 16-day study in a diabetes mouse model (mean±SEM; *** means (A) p<0.0002, (B) p<0.0003 or (C) p<0.0026 using an unpaired t-test).

FIG. 11 illustrates a dose-response curve for daily dosing of a comparator for the derivatives of the invention (herein identified as Comparator 9) (three different doses) compared to Comparator 4 and hFc-hIL-22 on blood glucose in a 13-day study in a diabetes mouse model (mean±SEM).

FIGS. 12A and B illustrate the effect of Comparators 4 and 9 in preventing liver injury in an acetaminophen (APAP)-induced liver injury mouse model, as evidenced by plasma levels of two different liver enzymes. Using Dunnett's test one-factor linear model, * means p<0.05 and ** means p<0.01 compared to vehicle+APAP.

FIG. 13 illustrates the effect of Comparators 4 and 9, (A) in preventing apoptosis and (B) on cellular proliferation, in an APAP-induced liver injury mouse model. NS means non-significant.

FIG. 14 illustrates the effect of Comparator 9 in preventing and/or reducing (A) lung inflammation, and (B) and (C) lung fibrosis, in a bleomycin-induced lung injury rat model, compared to prednisolone.

FIG. 15 illustrates the effect of Comparator 9 in preventing colon inflammation in a dextran sulfate sodium (DSS)-induced colitis mouse model. **** means p<0.0001 compared to vehicle (containing DSS).

FIG. 16 illustrates the effect of Comparator 9 compared to hFc-hIL-22 in preventing mucosal epithelial wounding in the DSS-induced colitis mouse model. Magnification 4×, scale bar=500 μm.

FIG. 17 illustrates plasma Regenerating Islet Derived Protein 3 Gamma (Reg3g) levels in the DSS-induced colitis mouse model, as a measure of target engagement (Reg3g is a target engagement marker of IL-22).

FIGS. 18A and B illustrate the effect of Comparator 4 in preventing liver injury in a Concanavalin A (ConA)-induced liver injury mouse model, as evidenced by serum levels of two different liver enzymes.

### DETAILED DESCRIPTION

In what follows, Greek letters are represented by their symbol rather than their written name. For example, α=alpha, ε=epsilon, γ=gamma and μ=mu. Amino acid residues may be identified by their full name, three-letter code or one-letter code, all of which are fully equivalent.

The term “derivative of IL-22”, as used herein, refers to an IL-22 protein having a covalently attached fatty acid, wherein the fatty acid is a monoacid. The term encompasses both derivatives in which the fatty monoacid is covalently attached to the IL-22 protein directly and those in which the covalent attachment is by a linker.

The covalent attachment of fatty acids is a proven technology for half-life extension of peptides and proteins and is a way of subtending a fatty acid from the peptide or protein. It is known from marketed products for types 1 and 2 diabetes, such as insulins Levemir® (detemir) and Tresiba® (degludec), and glucagon-like peptide-1 (GLP-1) derivatives Victoza® (liraglutide) and Ozempic® (semaglutide).

Fatty acid attachment enables binding to albumin, thereby preventing renal excretion and providing some steric protection against proteolysis. Advantageously, it offers a minimal modification to IL-22 compared to Fc fusion or PEGylation. In this regard, whilst Fc fusion and PEGylation aim to increase the size of IL-22 beyond the threshold for renal clearance, derivatives comprising a fatty monoacid covalently attached to an IL-22 protein retain a small size similar to that of the IL-22 protein. Thus, as the fatty monoacid attachment is a minimal modification, the resultant derivative is believed to maintain native-like properties including distribution, diffusion rate and receptor engagement (binding, activation and trafficking) and minimise immunogenicity risk.

As above, fatty acid attachment has proven therapeutic efficacy in insulin and GLP-1 derivatives for diabetes. However, IL-22 is a very different protein in terms of its size, sequence and biological properties. It was therefore counterintuitive to the inventors that fatty monoacids could be covalently attached to IL-22 whilst maintaining therapeutic effect. It was particularly surprising that such a minimal modification to IL-22 could result in high potency (identical to or close to hIL-22) combined with a prolonged circulatory half-life or prolonged half-life in biological fluids (e.g. plasma or intestinal fluid) or tissue preparations.

In a first aspect, therefore, the invention relates to a derivative of IL-22 comprising a fatty acid covalently attached to an IL-22 protein, wherein the fatty acid is a monoacid. The fatty monoacid may be covalently attached to the IL-22 protein directly or via a linker, which itself can be devised of various subunits. The term, “IL-22 protein”, as used herein, can mean a native IL-22 protein, such as hIL-22, or a variant thereof. A “variant” can be a protein having a similar amino acid sequence to that of the native protein, as further defined herein.

In nature, human IL-22 protein is synthesised with a signal peptide of 33 amino acids for secretion. The mature human IL-22 protein (i.e. hIL-22) is 146 amino acids in length and has 80.8% sequence identity with murine IL-22 (the latter being 147 amino acids in length). The amino acid sequence of hIL-22 is identified herein as SEQ ID NO. 1. Like other IL-10 family members, the IL-22 structure contains six α-helices (referred to as helices A to F).

The derivatives of the invention may thus have the native amino acid sequence of hIL-22. Alternatively, they may have one or more amino acid sequence variations within the native sequence. They may additionally or alternatively include one or more amino acid sequence variations relative to (i.e. outside) the native sequence. Thus, in an embodiment, the derivative comprises a fatty monoacid covalently attached to hIL-22 or a variant thereof.

Expressions such as “within”, “relative to”, “corresponding to” and “equivalent to” are used herein to characterise the site of change and/or covalent attachment of a fatty monoacid in an IL-22 protein by reference to the sequence of the native protein, e.g. hIL-22. In SEQ ID NO. 1, the first amino acid residue of hIL-22 (alanine (Ala)) is assigned position 1.

Thus, a variation within the sequence of hIL-22 is a variation to any of residue numbers 1-146 in SEQ ID NO. 1. For example, a Glu substitution for the native Asp at residue 10 in hIL-22 is represented herein as “D10E”. If the derivative also has a fatty monoacid covalently attached at position 10, it is herein referred to as attachment at residue “10E”.

A variation relative to the sequence of hIL-22, however, is a variation external to residue numbers 1-146 in SEQ ID NO. 1. For example, Derivative 1 as defined herein includes an N-terminal peptide of three amino acids in length. The residues in the N-terminal peptide are numbered negatively, starting from the residue attached to residue 1 in hIL-22, i.e. the first residue in the N-terminal peptide that is attached to residue 1 in hIL-22 is denoted “−1”. Derivative 1 has a fatty monoacid covalently attached at the first residue in hIL-22, i.e. position 4 in the sequence listing for Derivative 1, which is Cys and thus herein referred to as “4C”. By way of example, however, if the fatty monoacid had been covalently attached at the third residue in the N-terminal peptide starting from position −1, this being Glu, the covalent attachment site for Derivative 1 would have been herein referred to as “-3G”. Naturally, however, the numbering used in the sequence listing for Derivative 1 starts from 1, in accordance with WIPO Standard ST.25; as such, position 1 in the sequence listing for Derivative 1 is actually residue −3 as referred to herein.

Two, three, four, five or more variations may be made within the native sequence to form the derivatives of the invention. For example, more than 10, 15, 20, 25, 50, 75, 100 or even more than 125 variations may be made in this regard. Any of residues 1-146 in the native sequence may be varied. Exemplary residues for variation are residues 1, 2, 3, 4, 5, 6, 7, 8, 9, 10, 11, 12, 13, 14, 15, 16, 17, 18, 19, 20, 21, 22, 24, 25, 26, 27, 29, 30, 32, 33, 34, 35, 36, 37, 38, 39, 40, 41, 42, 44, 45, 47, 48, 49, 50, 51, 52, 53, 54, 55, 56, 58, 59, 61, 62, 63, 64, 65, 67, 68, 69, 70, 71, 72, 73, 74, 75, 77, 78, 79, 82, 83, 84, 86, 88, 90, 91, 92, 93, 94, 95, 96, 97, 98, 99, 100, 102, 103, 104, 105, 106, 107, 108, 109, 110, 111, 112, 113, 114, 115, 116, 117, 118, 119, 120, 121, 122, 123, 124, 126, 127, 128, 129, 130, 132, 133, 134, 135, 137, 138, 139, 141, 143, 144, 145 and/or 146 in hIL-22. Variation at residues 1, 21, 35, 64, 113 and/or 114 is particularly advantageous. Variation at residues 95 and/or 106 is also advantageous.

The variations within the native sequence are typically amino acid substitutions. The term “substitution”, as used herein, can mean the replacement of an amino acid in the native protein with another. They may be conservative or non-conservative substitutions. Exemplary substitutions are A1C, A1G, A1H, P2C, P2H, I3C, I3H, I3V, S4H, S4N, S5H, S5T, H6C, H6R, C7G, R8G, R8K, L9S, D10E, D10S, K11C, K11G, K11V, S12C, N13C, N13G, F14S, Q15C, Q15E, Q16V, P17L, Y18F, I19Q, T20V, N21C, N21D, N21Q, R22S, F24H, M25E, M25L, L26S, A27L, E29P, A30Q, L32C, L32R, A33C, A33N, D34F, N35C, N35D, N35H, N35Q, N36Q, T37C, T37I, D38L, V39Q, R40W, L41Q, I42P, E44R, K45A, F47T, H48G, H48R, G49N, V50S, S51C, M52A, M52C, M52L, M52V, S53C, S53K, S53Y, E54D, E54F, R55Q, R55V, C56Q, L58K, M59I, Q61E, V62D, L63C, N64C, N64D, N64Q, N64W, F65G, L67Q, E69D, E69L, V70S, L71C, F72D, F72L, P73C, P73L, Q74T, R77I, F78Q, Q79E, M82Y, Q83G, E84R, V86A, F88N, A90P, A90T, R91C, R91K, R91Y, L92R, S93Y, N94C, N94Q, R95K, R95Q, L96E, S97K, T98C, T98N, T98S, C99V, H100S, E102S, G103D, D104Y, D105Y, L106E, L106Q, H107L, H107N, 1108L, Q109Y, R110C, R110K, N111K, V112E, Q113C, Q113R, K114C, K114R, L115V, K116Y, D117E, T118G, V119A, K120H, K121R, L122A, G123V, G126Y, E127C, 1128V, K129V, G132Y, E133Q, L134P, D135M, L137D, F138R, M139L, M139R, L141Q, N143S, A144E, C145E, 1146R and/or I146V. Advantageously, the substitution may be selected from the group consisting of A1C, A1G, A1H, N21C, N21D, N21Q, N35C, N35D, N35H, N35Q, N64C, N64D, N64Q, N64W, Q113C, Q113R, K114C and K114R. Advantageously the substitution may be R95C or L106C. Surprisingly, substitutions as employed in the invention do not adversely affect IL-22 activity.

Particular combinations of substitutions include (i) A1G, N21D, N35D and N64D; (ii) A1G, 13V, S4N, S5T, H6R, R8K, D10E, K11V, T20V, H48R, M52A, S53K, E54D, R55Q, E69D, F72L, A90T, R91K, R95Q, T98S, E102S, L106Q, H107N, R110K, Q113R, K114R, D117E and I146V; (iii) A1G, 13V, S4N, SST, H6R, R8K, D10E, K11V, T20V, H48R, M52A, S53K, E54D, R55Q, E69D, F72L, A90T, R91K, R95Q, T98S, E102S, L106Q, H107N, R110K, Q113R, K114R, D117E and I146V; (iv) A1G, N35Q and N64Q; (v) A1G and N64C; (vi) A1G and Q113C; (vii) A1G and K114C; (viii) A1G and M25L; (ix) A1G and M52L; (x) A1G and M139L; (xi) A1G and N36Q; (xii) A1G and D117E; (xiii) A1G and N21Q; (xiv) A1G and N35Q; (xv) A1G and N64Q; (xvi) A1G, N21Q and N35Q; (xvii) A1G, N21Q and N64Q; (xviii) A1G, N21Q, N35Q and N64Q; (xix) A1G and K11C; (xx) A1G and N13C; (xxi) N35Q and N64Q; (xxii) A1C, N35Q and N64Q; (xxiii) H6C, N35Q and N64Q; (xxiv) I3C, N35Q and N64Q; (xxv) P2C, N35Q and N64Q; (xxvi) L32C, N35Q and N64Q; (xxvii) N35Q, M52C and N64Q; (xxviii) N13C, N35Q and N64Q; (xxix) N21C, N35Q and N64Q; (xxx) N35Q, N64Q and N94C; (xxxi) N35Q, N64Q and P73C; (xxxii) N35Q, N64Q and Q113C; (xxxiii) N35Q, N64Q and R91C; (xxxiv) N35Q, N64Q and R110C; (xxxv) S12C, N35Q and N64Q; (xxxvi) N35Q, S51C and N64Q; (xxxvii) N35Q, S53C and N64Q; (xxxviii) N35Q, T37C and N64Q; (xxxix) N35Q, N64Q and T98C; (xxxx) Q15C, N35Q and N64Q; (xxxxi) N35C and N64Q; (xxxxii) H6C, N35Q and N64Q; (xxxxiii) A33C, N35Q and N64Q; and (xxxxiv) A1H, P2H, 13H, S4H, S5H, C7G, R8G, L9S, D10S, K11G, N13G, F14S, Q15E, Q16V, P17L, 18F, Y19Q, N21Q, R22S, F24H, M25E, L26S, A27L, E29P, A30Q, L32R, A33N, D34F, N35H, T37I, D38L, V39Q, R40W, L41Q, 142P, E44R, K45A, F47T, H48G, G49N, V50S, M52V, S53Y, E54F, R55V, C56Q, L58K, M59I, Q61E, V62D, L63C, N64W, F65G, L67Q, E69L, V70S, L71C, F72D, P73L, Q74T, R771, F78Q, Q79E, M82Y, Q83G, E84R, V86A, F88N, A90P, R91Y, L92R, S93Y, N94Q, R95K, L96E, S97K, T98N, C99V, H100S, G103D, D104Y, D105Y, L106E, H107L, 1108L, Q109Y, R111K, V112E, L115V, K116Y, D117E, T118G, V119A, K120H, K121R, L122A, G123V, G126Y, E127C, 1128V, K129V, G132Y, E133Q, L134P, D135M, L137D, F138R, M139R, L141Q, N143S, A144E, C145E and I146R. Other particular combinations of substitutions include (xxxxv) N35Q, N64Q and R95C; and (xxxxvi) N35Q, N64Q and L106C. Any and all combinations of substitutions are envisaged and form part of the invention.

A derivative of the first aspect may typically comprise an amino acid substitution whereby Cys is substituted for a native residue, optionally in any of the positions identified above, such as position 1, 2, 3, 6, 11, 12, 13, 15, 21, 32, 33, 35, 37, 51, 52, 53, 63, 64, 71, 73, 91, 94, 98, 110, 113, 114 and/or 127. Advantageously, the IL-22 protein included in a derivative of the first aspect comprises a Cys residue at position 1 of hIL-22 (such as in Derivative 1 and Derivative 2). In another preferred embodiment, the variant comprises a Cys residue at position 95 or 106 of hIL-22.

Alternatively, or in addition, the variations within the native sequence may be amino acid insertions. Up to five, 10, 15, 20, 25, 30, 35, 40, 45 or even up to 50 amino acids may be inserted within the native sequence. Trimers, pentamers, septamers, octamers, nonamers and 44-mers are particularly advantageous in this regard. Exemplary sequences are shown in Table 1. Insertions can be made at any location in the native sequence, but those in helix A (for example, at residue 30), loop CD (for example, at residue 75), helix D (for example, at residue 85) and/or helix F (for example, at residue 124) are preferred.

TABLE 1
Sequence of exemplary amino acid insertions

| n-mer | Exemplary amino acid sequence | SEQ ID NO. |
|---|---|---|
| Trimer | E-T-S | n/a |
| Pentamer | R-V-Q-F-Q or C-V-E-I-P | 2, 3 |
| Septamer | G-S-G-S-G-S-C | 4 |
| Octamer | I-E-A-L-T-P-H-S or | 5, 6 |
| | Y-G-Q-R-Q-W-K-N | |
| Nonamer | V-F-I-I-N-N-S-L-E | 7 |
| 44-mer | R-A-A-S-A-G-S-Y-S- | 8 |
| | E-W-S-M-T-P-R-F-T- | |
| | P-W-W-E-T-K-I-D-P- | |
| | P-V-M-N-I-T-Q-V-N- | |
| | G-S-L-L-V-I-L-H | |

The one, two, three, four, five or more variations within the native sequence may be independently selected from the group consisting of substitutions and insertions.

The variations within the native sequence may also or alternatively comprise one or more amino acid deletions within SEQ ID NO. 1.

The peptide may thus comprise up to five amino acid deletions. No more than three or two amino acid deletions are preferred. Said deletions may be present in separate (i.e. non-consecutive) positions, e.g., within SEQ ID NO: 1. The variations may also or alternatively be a deletion of two, three, four or five consecutive amino acids within SEQ ID NO:1, meaning that a series of up to five neighbouring amino acids may be deleted.

Sequence variations relative to the amino acid sequence of hIL-22, if present, typically include an extension, such as the addition of a peptide at the N-terminal end. The peptide may consist of up to five, 10, 15, 20, 25, 30, 35, 40, 45 or even up to 50 amino acids. Monomers, trimers, octamers, 13-mers, 15-mers, 16-mers, 21-mers, 28-mers are particularly advantageous in this regard. Exemplary sequences are shown in Table 2. Suitably, the IL-22 protein included in a derivative of the first aspect comprises an N-terminal G-P-G. In a particularly preferred example, the derivative of the first aspect comprises both a Cys residue at position 1 of hIL-22 (SEQ ID NO. 1) and an N-terminal G-P-G. This has been found to create a derivative with a very good half-life and potency (see Derivative 1 in Example 1).

TABLE 2
Sequence of exemplary N-terminal peptides

| n-mer | Exemplary amino acid sequence | SEQ ID NO. |
|---|---|---|
| Monomer | C, Gor M | n/a |
| Trimer | G-P-G | n/a |
| Pentamer | A-E-P-E-E | 9 |
| Hexamer | A-C-E-P-E-E | 10 |
| Octamer | G-P-A-C-E-P-E-E | 11 |
| 13-mer | G-G-S-S-G-S-G-S-E-V-L-F-Q | 12 |
| 13-mer | G-S-G-S-G-S-C-G-S-G-S-G-S | 13 |
| 14-mer | G-G-S-S-G-S-G-S-E-V-L-F- | 14 |
| | Q-G | |
| 15-mer | G-P-G-S-G-S-G-S-C-G-S-G- | 15 |
| | S-G-S | |
| 16-mer | G-G-S-S-G-S-G-S-E-V-L-F- | 16 |
| | Q-G-P-G | |
| 21-mer | G-G-S-S-G-S-G-S-E-V-L-F- | 17 |
| | Q-G-P-A-C-E-P-E-E | |
| 28-mer | G-G-S-S-G-S-G-S-E-V-L-F- | 18 |
| | Q-G-P-G-S-G-S-G-S-C-G-S- | |
| | G-S-G-S | |

In another advantageous embodiment, the IL-22 protein included in a derivative of the first aspect does not comprise an N-terminal G-P-G.

Sequence variations relative to the amino acid sequence of hIL-22, if present, may include the addition of a peptide at the C-terminal end. The peptide may consist of up to five, 10, 15, 20, 25, 30, 35, 40, 45 or even up to 50 amino acids. Exemplary C-terminal peptide sequences include those shown in Table 2 (for N-terminal peptides). A septamer is particularly advantageous in this regard, optionally having the amino acid sequence, G-S-G-S-G-S-C(SEQ ID NO. 19).

The derivatives of the invention may include both an N-terminal and a C-terminal peptide in addition to the native or variant hIL-22 amino acid sequence as herein described. Any combination of the N- and C-terminal peptides described herein is envisaged and expressly included in the invention.

It will be appreciated that the invention extends to any derivative of IL-22, which comprises a fatty acid covalently attached to hIL-22 or a variant thereof, wherein the fatty acid is a monoacid. The “variant” can be a protein having at least 10% sequence identity with hIL-22. For example, the variant may have a variation within hIL-22 and have at least 10% sequence identity with hIL-22. In an embodiment, the variant has at least 20%, or even at least 30%, sequence identity with hIL-22. The variant may have “substantially the amino acid sequence” of hIL-22, which can mean a sequence that has at least 40% sequence identity with the amino acid sequence of hIL-22. Accordingly, in an embodiment, a derivative of the first aspect has at least 50%, at least 60%, at least 70%, at least 80%, at least 90%, at least 95% or 100% amino acid sequence identity with hIL-22. Exemplary IL-22 protein variants, including that incorporated in the particular derivative of the invention disclosed in the experimental section, are set forth in SEQ ID NOs. 20-25.

The skilled technician will appreciate how to calculate the percentage identity between two amino acid sequences. An alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local versus global alignment, the pair-score matrix used (for example, BLOSUM62, PAM250, Gonnet etc.) and gap-penalty, for example, functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

Hence, it will be appreciated that the accurate alignment of amino acid sequences is a complex process. The popular multiple alignment program ClustalW [48,49] is a preferred way for generating multiple alignments of proteins in accordance with the invention.

Suitable parameters for ClustalW may be as follows: For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art.

Suitably, a derivative of the first aspect comprises 200 amino acids or less. For example, the derivative comprises less than 190, less than 180, less than 170, less than 160 or even less than 150 amino acids. Suitably, the derivative will comprise at least 146 amino acids, however, this being the number of amino acids in hIL-22. It may comprise at least 150 amino acids, at least 160 amino acids, at least 170 amino acids or even at least 180 amino acids. The derivatives of the invention can comprise proteins of any length within the above ranges, but they will typically be 146-180 amino acids in length.

The derivatives of the invention, whether having the native or a variant amino acid sequence, include a fatty acid covalently attached to the IL-22 protein, wherein the fatty acid is a monoacid. The fatty monoacid is typically covalently attached to the IL-22 protein by a linker. The fatty monoacid and linker are suitably connected to each other via an amide bond, and the linker is covalently attached to the IL-22 protein. The fatty monoacid and linker may thus be present as a side chain on the IL-22 protein. It was surprising to the inventors that a covalently attached fatty monoacid does not adversely affect IL-22 activity. It was particularly surprising that fatty monoacid attachment is associated with additional advantages, such as prolongation of half-life.

The fatty monoacid may be any suitable fatty monoacid. In particular, the fatty monoacid may be of Formula I:

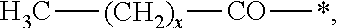

wherein x is an integer in the range of 10-18, optionally 12-18, 14-16 or 16-18, and * designates a point of attachment to the IL-22 protein or linker. It may be a C12, C14, C16, C18 or C20 monoacid. Advantageously, the fatty monoacid is a C16 or C18 monoacid, and most advantageously it is a C16 monoacid.

For example, —(CH₂)ₓ— in Formula I may be a straight alkylene in which x is 10. This fatty acid may be conveniently referred to as C12 monoacid, i.e. a fatty mono-carboxylic acid with 12 carbon atoms. Alternatively, —(CH₂)ₓ— in Formula I may be a straight alkylene in which x is 12. This fatty acid may be conveniently referred to as C14 monoacid, i.e. a fatty mono-carboxylic acid with 14 carbon atoms. In a similar fashion, —(CH₂)ₓ— in Formula I may be a straight alkylene in which x is 14 (C16 monoacid), 16 (C18 monoacid) or 18 (C20 monoacid).

Suitably, a derivative of the first aspect includes a C14, C16, C18 or C20 monoacid; more suitably, a C16 or C18 monoacid, and even more suitably a C16 monoacid.

The monoacid may be capable of forming non-covalent associations with albumin, thereby promoting circulation of the derivative in the blood stream. The shorter monoacids (e.g. C16 monoacid) have lower albumin affinity and thus a shorter half-life than the longer monoacids (e.g. C18 monoacid). However, they are still long acting derivatives with an expected half-life in man of over one day.

The monoacids are also lipophilic, which means they tend to bind to bio-membranes. This non-albumin dependent protraction through integration in bio-membranes may form a local reservoir, thus ensuring a prolonged local action. This could be hypothesised to provide an advantage in topical administration, oral administration, rectal suppositories or rectal foams or pulmonary inhalation.

Fatty acid attachment will, in itself, also stabilise the IL-22 protein against proteolytic degradation.

The derivatives of the first aspect may comprise particular combinations of a fatty monoacid and IL-22 protein. For example, a C14, C16, C18 or C20 monoacid may be attached to an IL-22 protein comprising a Cys residue at position 1 of hIL-22 and/or an N-terminal G-P-G. In one example, a derivative of the first aspect comprises a C16 monoacid and the IL-22 protein comprises both a Cys residue at position 1 of hIL-22 and an N-terminal G-P-G (such as in Derivative 1).

As above, the fatty monoacid is suitably connected to a linker, which is attached to the IL-22 protein. The linker may comprise several linker elements, including one or more amino acids such as one or more Glu and/or Lys residues. The linker may include an oxyethylene glycine unit or multiple linked oxyethylene glycine units, optionally 2-5 such units, advantageously 2 units. One or more OEG residues, C2DA and/or Ac groups may alternatively or additionally be included. The linker may comprise a Cys-reactive unit. A “Cys-reactive unit”, as used herein, can mean a functional unit that is able to react with the sulphur atom of a Cys to create a carbon-sulphur covalent bond. The Cys-reactive unit can have any of several forms, but suitably includes a carbon atom attached to a leaving group, which leaving group becomes displaced by the sulphur atom of the Cys during formation of the carbon-sulphur bond. The leaving group may be a halogen, optionally a bromine atom. This bromide leaving group can be alpha to an Ac functional group; advantageously it is a bromo-Ac functional group. The leaving group may alternatively be a functionalised hydroxyl group of the form mesylate or tosylate, or an unfunctionalised hydroxyl group. Further, the leaving group can be a maleimide or other functional group. Exemplary linkers include γGlu-OEG-OEG-C2DA-Ac, γGlu-γGlu-γGlu-γGlu-OEG-OEG-εLys-αAc and γGlu-OEG-OEG-εLys-αAc, but any suitable linker may be employed. The linker, γGlu-OEG-OEG-εLys-αAc, may be preferred.

The fatty monoacid, or linker, may be attached to any amino acid residue in the IL-22 protein. Exemplary in this regard are residues −7, −5, 1, 6, 33, 113, 114 and 153 in or relative to the hIL-22 amino acid sequence. Further exemplary residues are 95 and 106 in the hIL-22 amino acid sequence. The native residue is typically substituted with Cys or Lys to enable attachment of the fatty monoacid or linker. Alternatively the fatty monoacid or linker can be attached at a native Cys or Lys residue. Suitably, the fatty monoacid or linker is attached to a Cys residue substituted at position 1, 6, 33, 113 or 114 of hIL-22 or to a Cys residue at position −5, −7 or 153 relative to hIL-22. In particular, the fatty monoacid or linker may be attached to a Cys residue substituted at position 1 of hIL-22. The fatty monoacid or linker may be attached to a Cys residue substituted at position 95 or 106 of hIL-22.

The attachment of the fatty monoacid or linker to the IL-22 protein is a covalent attachment. For example, a Cys-reactive fatty monoacid or linker may be used to attach the fatty monoacid or linker to a Cys residue in the IL-22 protein. The fatty monoacid or linker may be covalently attached to the sulphur atom of the Cys residue via a thioether bond. Alternatively, a Lys-reactive fatty monoacid or linker may be used to attach the fatty monoacid or linker to a Lys residue in the IL-22 protein. The fatty monoacid or linker may alternatively be covalently attached to the free amine (—NH₂) group in the N-terminus of the IL-22 protein (irrespective of the amino acid in position 1). Attachment can proceed as with Cys attachment, albeit with sub-stoichiometric amounts of fatty monoacid or linker containing a suitable N-reactive species. The fatty monoacid or linker may be presented in the form of an aldehyde (the N-reactive species) and be covalently attached to the free amine employing a classically known reductive amination.

A derivative of the first aspect thus suitably comprises a C14, C16, C18 or C20 monoacid attached by a linker to a variant of hIL-22, wherein the variant optionally comprises an N-terminal G-P-G and a Cys residue at position 1 of hIL-22 and the linker is optionally attached to the Cys residue.

In an embodiment, the derivative comprises a C14, C16, C18 or C20 monoacid covalently attached by a linker to a variant of hIL-22, wherein the linker is γGlu-OEG-OEG-εLys-αAc, the variant comprises an N-terminal G-P-G and a Cys residue at position 1 of hIL-22 and the linker is optionally attached to said Cys residue.

In an embodiment, the derivative comprises a C16, C18 or C20 monoacid covalently attached by a linker to a variant of hIL-22, wherein the linker is γGlu-OEG-OEG-εLys-αAc, the variant comprises an N-terminal G-P-G and a Cys residue at position 1 of hIL-22 and the linker is optionally attached to said Cys residue.

In an embodiment, the derivative comprises a C16 or C18 monoacid covalently attached by a linker to a variant of hIL-22, wherein the linker is γGlu-OEG-OEG-εLys-αAc, the variant comprises an N-terminal G-P-G and a Cys residue at position 1 of hIL-22 and the linker is optionally attached to said Cys residue.

In an embodiment, the derivative comprises a C16 monoacid covalently attached by a linker to a variant of hIL-22, wherein the linker is γGlu-OEG-OEG-εLys-αAc, the variant comprises an N-terminal G-P-G and a Cys residue at position 1 of hIL-22 and the linker is optionally attached to said Cys residue.

In an embodiment, the derivative comprises a C14, C16, C18 or C20 monoacid covalently attached by a linker to a variant of hIL-22, wherein the variant does not comprise an N-terminal G-P-G but comprises a Cys residue at position 1 of hIL-22 and the linker is optionally attached to said Cys residue.

In an embodiment, the derivative comprises a C14, C16, C18 or C20 monoacid covalently attached by a linker to a variant of hIL-22, wherein the linker is γGlu-OEG-OEG-εLys-αAc, the variant does not comprise an N-terminal G-P-G but comprises a Cys residue at position 1 of hIL-22 and the linker is optionally attached to said Cys residue.

In an embodiment, the derivative comprises a C16, C18 or C20 monoacid covalently attached by a linker to a variant of hIL-22, wherein the linker is γGlu-OEG-OEG-εLys-αAc, the variant does not comprise an N-terminal G-P-G but comprises a Cys residue at position 1 of hIL-22 and the linker is optionally attached to said Cys residue.

In an embodiment, the derivative comprises a C16 or C18 monoacid covalently attached by a linker to a variant of hIL-22, wherein the linker is γGlu-OEG-OEG-εLys-αAc, the variant does not comprise an N-terminal G-P-G but comprises a Cys residue at position 1 of hIL-22 and the linker is optionally attached to said Cys residue.

In an embodiment, the derivative comprises a C16 monoacid covalently attached by a linker to a variant of hIL-22, wherein the linker is γGlu-OEG-OEG-εLys-αAc, the variant does not comprise an N-terminal G-P-G but comprises a Cys residue at position 1 of hIL-22 and the linker is optionally attached to said Cys residue.

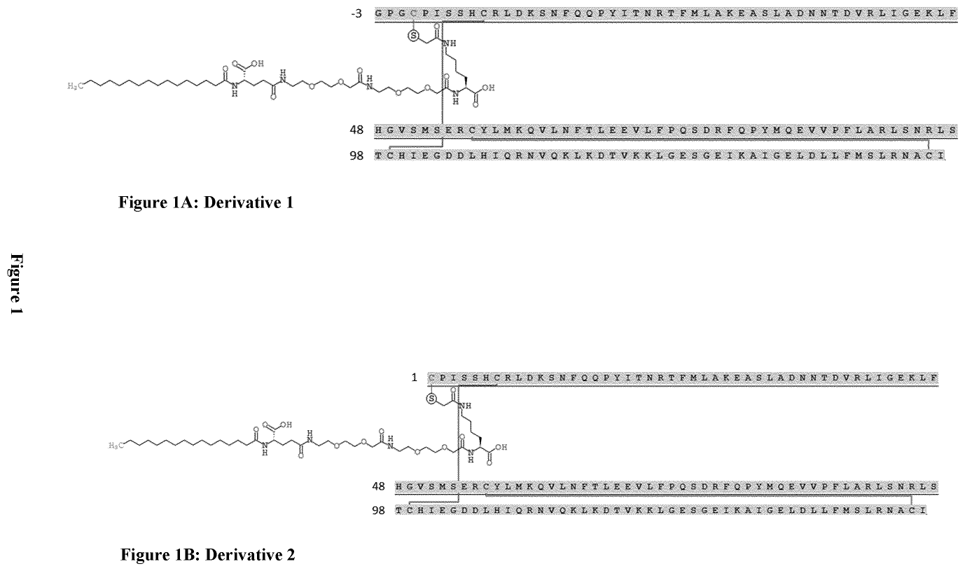

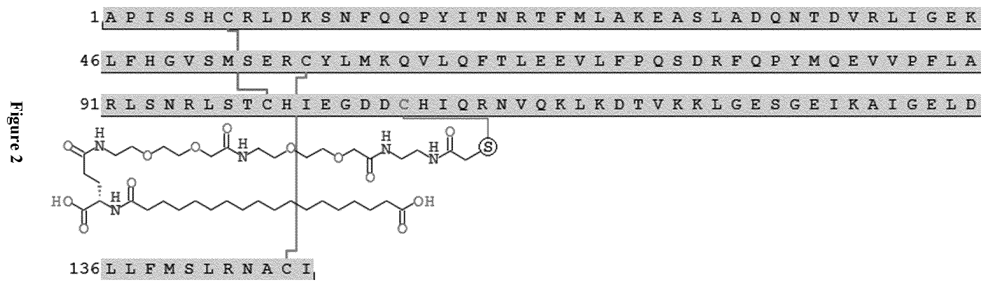

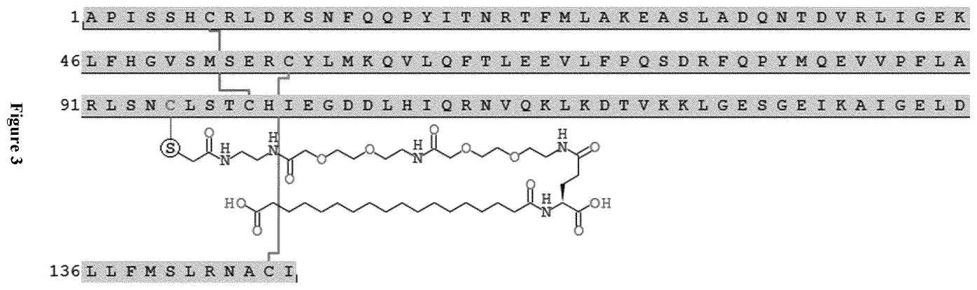

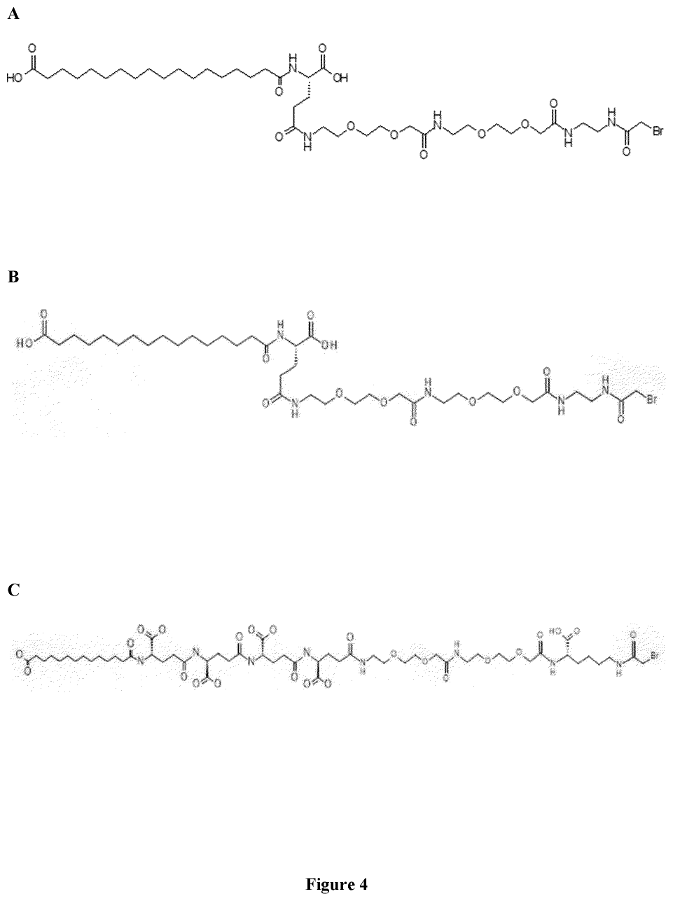

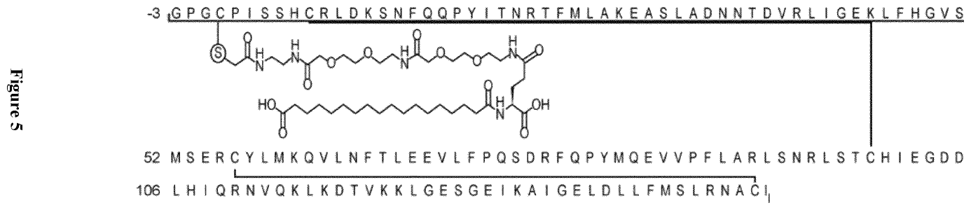

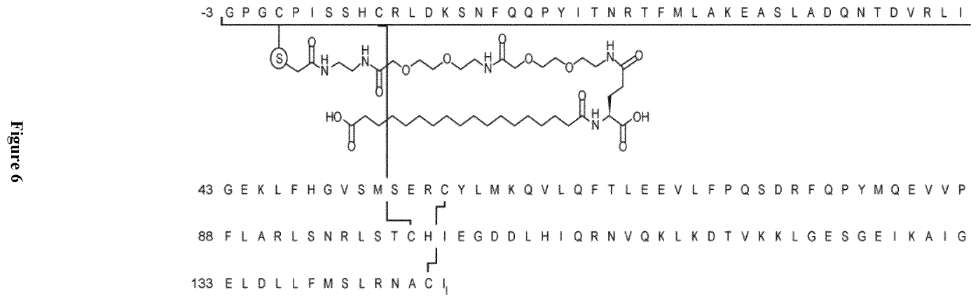

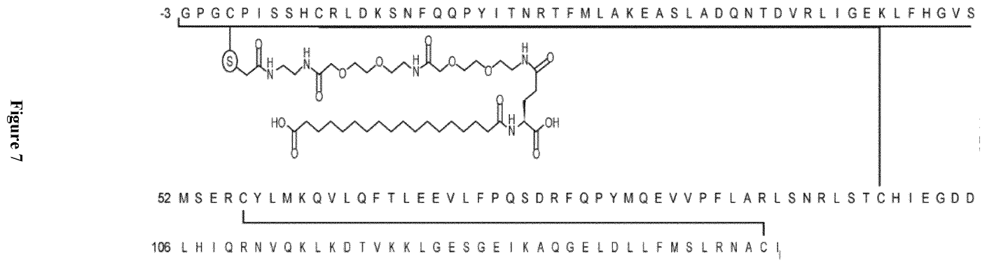

Exemplary derivatives of the first aspect comprise an IL-22 protein as set forth in any of SEQ ID NOs. 20-25 and 34. Particularly advantageous derivatives are shown in Table 3, illustrated in FIG. 1 and exemplified herein.

TABLE 3
Exemplary derivatives of IL-22

| | | | Covalent | | |
|---|---|---|---|---|---|
| | | SEQ ID | attachment | | Fatty |
| ID | Sequence variations | NO. | site | Linker | acid |
| Derivative 1 | A1C substitution & | 20 | 1C | γGlu-OEG- | C16 monoacid |
| | G-P-G N-terminal | | | OEG-εLys-αAc | |
| | peptide | | | | |
| Derivative 2 | A1C substitution | 34 | 1C | γGlu-OEG- | C16 monoacid |
| | | | | OEG-εLys-αAc | |

The derivatives of the invention may exist in different stereoisomeric forms and the invention relates to all of these.

According to a second aspect of the invention, there is provided a process for preparing a derivative of the first aspect comprising covalently attaching a fatty monoacid to an IL-22 protein.

The process may be used to produce any of the different derivatives of IL-22 described or envisaged herein, but it is particularly advantageous when a fatty monoacid is covalently attached to a variant IL-22 protein. Thus, in an embodiment, the IL-22 protein employed in the second aspect is a substituted form of hIL-22, optionally substituted at position 1, 21, 35, 64, 113 and/or 114. Exemplary substitutions include A1C, A1G, A1H, N21C, N21D, N21Q, N35C, N35D, N35H, N35Q, N64C, N64D, N64Q, N64W, Q113C, Q113R, K114C and/or K114R. Preferably, the IL-22 protein is substituted with a Cys residue at position 1.

The fatty monoacid can be obtained by any means known in the art, including recombinant means. Suitable fatty monoacids are commercially available or readily derived from available starting materials using standard chemical synthesis.

The IL-22 protein can be obtained by any means known in the art, including recombinant means. The production of recombinant hIL-22 has been previously described and is well-known in the art. Desired variant IL-22 proteins can be produced in a similar manner. An experienced investigator in the field would be readily able to identify suitable nucleic acid sequences that encode the desired variant IL-22 proteins. The skilled person would hence be readily able to execute this part of the invention, based upon the existing knowledge in the art. Suitably, the IL-22 proteins are produced in mammalian systems, such as in Chinese hamster ovary (CHO) cells, using standard techniques. A polyhistidine tag (His-tag) may be employed to aid affinity purification of the recombinant proteins.

In this regard, IL-22 proteins as used in the invention can be prepared using a post expression cleavable His-tag—an N- or C-terminal addition of less than 10, preferably six, histidine residues that can be purified by affinity to a nickel column. The His-tag is linked to the N- or C-terminal of the protein via a linker that can be digested by a known protease to leave the free IL-22 protein. The cleavable His-tag can have the amino acid sequence, HHHHHHGGSSGSGSEVLFQ (SEQ ID NO. 26), and the protease-cleavable linker can be a tobacco etch virus (TEV) linker, whose consensus sequence for the native cut sites is ENLYFQIS (SEQ ID NO. 27), where ‘\’ denotes the cleaved peptide bond or a human rhinovirus-14 3C (HRV14-3C) protease cleavable linker with EVLFQ consensus cleavage site. Cleavage may be achieved by incubating approximately 10 μg protease with 2.5 μg protein and 10 mM 2-mercaptoethanol at room temperature for 4 h.

To further illustrate the invention, a representative process for protein preparation is provided as follows. The process involves preparing a plasmid DNA that encodes the desired amino acid sequence of the IL-22 protein. This plasmid can be transiently transfected into a cell line, for example CHO-K1, which is allowed to grow in a relevant medium before growth is increased by the addition of a known enhancer. The secreted IL-22 protein can then be harvested through known methods of centrifugation and sterile filtration before the protein is purified on a nickel column. Following concentration and buffer exchange the His-tag is removed using a HRV14-3C protease before alkylation with a fatty monoacid (described further below) and final purification and buffer exchange. Analysis of the final product using SDS-PAGE, size exclusion chromatography or liquid chromatography with tandem mass spectrometry (LC-MS-MS) with, or without, deglycosylation can be used to ensure the quality of the final product.

The fatty monoacid can be covalently attached to the IL-22 protein either directly or using a linker as described for the first aspect. The linker can be obtained by any means known in the art. Methods for preparing the fatty monoacid and linker would be known to the skilled person. By way of example, however, a representative method for preparing a fatty diacid and linker, if employed, is described in US Patent Publication No. US 2018/0140673. The skilled person would know how to replace the disclosed diacids with monoacids.

Covalent attachment of the fatty monoacid or linker to the IL-22 protein may thus be carried out using standard procedures in the art. The linker, if employed, thus enables covalent attachment of the IL-22 protein to the fatty monoacid. By way of a non-limiting example, a Cys-reactive fatty monoacid or linker may be reacted with the sulphur atom of a Cys residue in the IL-22 protein, so forming a thioether bond. Suitable conditions for the covalent attachment step may be exemplified as follows: Tris in water is added to IL-22 protein (70 mg) in Tris and NaCl-buffer (1.35 mg/ml), to adjust to pH 8. Bis(p-sulfonatophenyl)-phenylphosphine dihydrate dipotassium (BSPP) salt (12 mg), dissolved in water, is added and stirred gently for four hours at room temperature. 15-{(S)-1-Carboxy-3-[2-(2-{[2-(2-{[2-(2-bromoacetylamino)-ethylcarbamoyl]ethoxy}ethoxy)ethylcarbamoyl]methoxy}ethoxy)ethylcarbamoyl]propyl-carbamoyl}pentadecanoic acid (19 mg, 0.022 mmol) in ethanol (0.5 ml) is added and the mixture stirred gently overnight. MiliQ water (150 ml) is added to lower the conductivity to 2.5 mS/cm. The mixture is then purified using anion exchange on a MonoQ 10/100 GL column using binding buffer (20 mM Tris, pH 8.0), elution buffer (20 mM Tris, 500 mM NaCl, pH 8.0), flow 6 ml and a gradient of 0-80% elution buffer over 60 column volumes.

The derivatives of the invention may be purified using any suitable procedure known in the art, such as chromatography, electrophoresis, differential solubility or extraction.

As described herein, the inventors were surprised to find that fatty monoacids could be covalently attached to an IL-22 protein whilst maintaining biological activity. It was particularly surprising that such a minimal modification to IL-22 could result in high potency (close to hIL-22) combined with a very long circulatory half-life. This particular combination of properties may be highly desirable.

The potency of the derivatives may be determined in an in vitro assay with whole cells expressing human IL-22 receptors. For example, the response of the human IL-22 receptors may be measured using baby hamster kidney (BHK) cells overexpressing IL-22R1, IL-10R2 and a phospho-STAT3 (pSTAT3) responsive reporter gene. Alternatively, HepG2 cells endogenously expressing the IL-22 receptor may be used. Activation of the receptors leads to activation of the STAT3 signaling pathway, which can be measured using a luciferase reporter gene with a STAT3-induced promoter or by assaying pSTAT3, for example. In vivo potency may be determined in animal models or in clinical trials, as is known in the art.

The half maximal effective concentration (EC₅₀) value is often used as a measure of the potency of a drug. As this represents the concentration of drug required to produce 50% of the maximal effect, the lower the EC₅₀ value, the better the potency. The derivatives of the invention suitably have a potency (EC₅₀ value) measured using IL-22 receptor-mediated STAT3 activation in cells of below 1.5 nM, below 1.25 nM, below 1 nM, below 0.75 nM, below 0.5 nM, below 0.25 nM or even below 0.1 nM (e.g. determined as described in Example 1). The derivatives of the invention suitably have a potency (EC₅₀ value) measured by assaying pSTAT3 in cells of below 15 nM, below 12 nM, below 10 nM, below 7 nM or even below 5 nM.

Advantageously, the potency of the derivatives of IL-22 may be higher than that of IL-22-Fc fusions. For example, Genentech has reported a 34-fold reduction in in vitro potency for its IL-22-Fc fusion, UTTR1147A, compared to hIL-22 (Stefanich et al., Biochem Pharmacol, 2018, 152:224-235). By contrast, covalent attachment of a fatty monoacid to hIL-22 has been shown to cause only a minimal reduction in potency (see Derivative 1 in Example 1 and Derivative 2 in Example 12). Whilst both IL-22-Fc fusions and the derivatives of the present invention may be comparable in terms of their improved half-life over hIL-22 and biological function in at least some settings, the derivatives of the invention may have the additional advantage of minimal loss of potency.

The circulatory elimination half-life (T_{1/2}) of the derivatives may be determined in vivo by administering the derivatives subcutaneously or intravenously in a suitable animal model, such as a mouse, rat or minipig. suitable methods will be known to the skilled person. By way of a non-limiting example, the derivatives of the first aspect have a circulatory half-life after subcutaneous or intravenous administration to mice of at least one hour, at least three hours, at least five hours or even at least eight hours. The derivatives may have a circulatory half-life after subcutaneous or intravenous administration to rats of at least three hours, at least five hours, at least eight hours, at least 10 hours or even at least 13 hours. The derivatives may have a circulatory half-life after subcutaneous or intravenous administration to minipigs of at least 25 hours, at least 40 hours, at least 70 hours or even at least 100 hours.

The inventors have also found that the derivatives of the invention are absorbed rapidly in vivo. Advantageously, absorption of the derivatives following subcutaneous dosing may occur faster than that of IL-22-Fc fusions. Mean absorption time is an accurate parameter for measuring uptake because it is independent of dose and maximum plasma concentration following drug administration. It can be calculated based upon mean residence time, i.e. the time that a drug spends in the body prior to elimination once absorption has been completed. The derivatives of the invention suitably have a mean absorption time in pigs of below 100 h, below 90 h, below 80 h, below 70 h or even below 60 h.

The derivatives of the invention also have good biophysical properties, such as high physical stability and/or solubility, which may be measured using standard methods in the art. In fact, monoacid derivatised variants have significantly altered biophysical properties compared to hIL-22, which improves their therapeutic potential. Of note, they are stabilised against proteolytical breakdown and renal clearance through binding to albumin and interaction with lipid membranes, for example. Importantly, the inventors have found that conjugation of monoacid-containing fatty acid moieties can be carried out at select sites of the IL-22 protein backbone, without compromising the activity of the conjugated derivative, as measured in vitro (see Example 1).

Therefore, according to a third aspect of the invention, there is provided a pharmaceutical composition comprising a derivative of the first aspect and a pharmaceutically acceptable vehicle. The pharmaceutical composition may be suitable for administration by inhalation, by injection, topically, orally or ocularly, optionally wherein the injection is intraperitoneal, subcutaneous or intravenous, as further described herein. In one embodiment subcutaneous administration is preferred. In one embodiment intravenous administration is preferred. In one embodiment oral administration is preferred. In one embodiment topical administration is preferred.

A pharmaceutical composition of the third aspect may comprise any of the different derivatives of IL-22 described or envisaged herein. Suitably, it comprises the derivative of IL-22 identified herein as Derivative 1. It may comprise the derivative of IL-22 identified herein as Derivative 2.

A derivative of the first aspect, or a pharmaceutical composition of the third aspect, will suitably demonstrate increased circulatory elimination half-life compared to hIL-22. Advantageously it will demonstrate increased circulatory elimination half-life compared to hIL-22 by at least 50%, at least 75%, at least 100% or more.

The pharmaceutical compositions of the third aspect may be prepared by combining a therapeutically effective amount of a derivative of the first aspect with a pharmaceutically acceptable vehicle. The formulation of pharmaceutically active ingredients with various excipients is known in the art.

A “therapeutically effective amount” of a derivative of the first aspect is any amount which, when administered to a subject, is the amount of derivative that is needed to treat the disease, disorder or condition or produce the desired effect.

For example, the therapeutically effective amount of derivative used may be from about 0.001 mg to about 1000 mg, and preferably from about 0.01 mg to about 500 mg. It is preferred that the amount of derivative is an amount from about 0.1 mg to about 100 mg, and most preferably from about 0.5 mg to about 50 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid; optionally the composition may be in the form of a powder for resuspension. A solid pharmaceutically acceptable vehicle may include one or more substances which may also act as flavouring agents, lubricants, solubilisers, suspending agents, dyes, fillers, glidants, inert binders, preservatives or dyes. The vehicle may also be an encapsulating material. In powders, the vehicle is a finely divided solid that is in admixture with the finely divided derivatives according to the invention. The powders preferably contain up to 99% derivative. Suitable solid vehicles include, for example calcium phosphate, magnesium stearate, talc, sugars, lactose, dextrin, starch, gelatin, cellulose and ion exchange resins.

In another embodiment, the pharmaceutical vehicle may be a gel and the composition may be in the form of a cream or the like.

However, the pharmaceutical vehicle may be a liquid; optionally the pharmaceutical composition is in the form of a solution. Liquid vehicles are used in preparing solutions, suspensions, emulsions, syrups, elixirs and pressurised compositions. The derivative according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilisers or osmo-regulators. Suitable examples of liquid vehicles for parenteral administration include water (partially containing additives as above, for example, cellulose derivatives, preferably sodium carboxymethyl cellulose solution), alcohols (including monohydric alcohols and polyhydric alcohols, for example, glycols) and their derivatives, and oils (for example, fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurised compositions can be a halogenated hydrocarbon or other pharmaceutically acceptable propellant.

The process for preparing a pharmaceutical composition of the invention may thus comprise the usual steps that are standard in the art.

According to a fourth aspect of the invention, therefore, there is provided a derivative of the first aspect, or a pharmaceutical composition of the third aspect, for use in therapy. A method of treating a subject with a derivative of the invention, or a pharmaceutical composition comprising the same, is also provided. Any of the different derivatives of IL-22 described or envisaged herein are expressly included in these aspects of the invention.

Terms such as “treating” and “therapy”, as used herein, expressly include the treatment, amelioration or prevention of a disease, disorder or condition.

The derivative of IL-22 or pharmaceutical composition comprising the same may be administered directly into a subject to be treated. The derivative or pharmaceutical composition may be administered by any means, including by inhalation, by injection, topically, orally, rectally or ocularly. When administered by inhalation, it may be via the nose or the mouth. Preferably, the derivative or pharmaceutical composition is administered by injection, typically subcutaneously or intravenously. As aforementioned, the lipophilicity of the monoacid derivatives of the invention may also be advantageous in topical administration, oral administration, rectal administration (e.g. suppositories or foams) or pulmonary inhalation. The derivatives therefore have a clear advantage over Fc fusions in their flexibility of administration (e.g. by injection, by inhalation, topical application, rectal or oral administration or ocular delivery) because of their smaller size and higher potency. It will be appreciated that administration, into a subject to be treated, of a derivative of the invention will result in the increased circulation time compared to hIL-22, and that this will aide in treating a disease, disorder or condition. As above, “treating” also includes ameliorating and preventing a disease, disorder or condition.

Liquid pharmaceutical compositions, which are sterile solutions or suspensions, can be utilised by, for example, intramuscular, intrathecal, epidural, intraperitoneal and particularly subcutaneous or intravenous injection. The derivative may be prepared as a sterile solid composition that may be dissolved or suspended at the time of administration using sterile water, saline or other appropriate sterile injectable medium.

Forms useful for inhalation include sterile solutions, emulsions and suspensions. Alternatively the derivatives may be administered in the form of a fine powder or aerosol via a Dischaler® or Turbohaler®. Nasal inhalations may suitably be in the form of a fine powder or aerosol nasal spray or modified Dischaler® or Turbohaler®.

Topical formulations include solutions, creams, foams, gels, lotions, ointments, pastes, tinctures and powders. They may be epicutaneous, i.e. applied directly to the skin, or applied to mucous membranes.

Oral administration may be suitably via a tablet, a capsule or a liquid suspension or emulsion. suitable examples of liquid vehicles for oral administration include water (partially containing additives, for example, cellulose derivatives, preferably sodium carboxymethyl cellulose solution), alcohols (including monohydric alcohols and polyhydric alcohols, for example, glycols) and their derivatives, and oils (for example, fractionated coconut oil and arachis oil). The derivatives of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. Solutions, syrups and elixirs also form part of the invention. The derivatives according to the invention can also be administered orally in solid composition form. Solid compositions suitable for oral administration include pills, capsules, granules, tablets and powders.

Rectal administration may suitably be via a suppository or foam.

Formulations for ocular administration are typically solutions, suspensions and ointments for topical application, e.g. in the form of eye drops. Alternatively sterile solutions or suspensions can be utilised by intraocular injection. The derivative may be prepared as a sterile solid composition that may be dissolved or suspended at the time of administration using sterile water, saline or other appropriate sterile injectable medium. The formulation may be for subconjunctival, intravitreal, retrobulbar or intracameral injection.

A derivative or pharmaceutical composition of the invention may be administered to any subject in need thereof. A “subject”, as used herein, may be a vertebrate, mammal or domestic animal. Hence, derivatives and compositions according to the invention may be used to treat any mammal, for example livestock (for example, a horse), pets, or may be used in other veterinary applications. Most preferably, the subject is a human being. The derivatives and compositions need not only be administered to those already showing signs of a disease, disorder or condition. Rather, they can be administered to apparently healthy subjects as a purely preventative measure against the possibility of such a disease, disorder or condition in future.

It will be appreciated that derivatives of IL-22 and compositions according to the invention may be used in a monotherapy (i.e. the sole use of that derivative or composition), for treating a disease, disorder or condition. Alternatively, derivatives and compositions according to the invention may be used as an adjunct to, or in combination with, known therapies for treating a disease, disorder or condition.

It will be appreciated that the amount of the derivative of IL-22 that is required is determined by its biological activity, half-life and bioavailability, which in turn depends on the mode of administration, the physiochemical properties of the derivative and composition, and whether it is being used as a monotherapy or in a combined therapy. The frequency of administration will also be influenced by the half-life of the derivative within the subject being treated. Optimal dosages to be administered may be determined by those skilled in the art, and will vary with the particular derivative in use, the strength of the pharmaceutical composition, the mode of administration, and the advancement of the disease, disorder or condition. Additional factors depending on the particular subject being treated will result in a need to adjust dosages, including subject age, weight, gender, diet and time of administration.

Generally, a daily dose of between 0.001 μg/kg of body weight and 10 mg/kg of body weight of derivative of IL-22 according to the invention may be used for treating a disease, disorder or condition, depending upon which derivative or composition is used. More preferably, the daily dose is between 0.01 μg/kg of body weight and 1 mg/kg of body weight, more preferably between 0.1 μg/kg and 500 μg/kg body weight, and most preferably between approximately 0.1 μg/kg and 100 μg/kg body weight.

The derivative of IL-22 or composition may be administered before, during or after onset of the disease, disorder or condition. Daily doses may be given as a single administration (for example, a single daily injection). Alternatively, the derivative or composition may require administration twice or more times during a day. As an example, derivatives may be administered as two (or more depending upon the severity of the disease, disorder or condition being treated) daily doses of between 0.07 μg and 700 mg (i.e. assuming a body weight of 70 kg). A patient receiving treatment may take a first dose upon waking and then a second dose in the evening (if on a two-dose regime) or at 3- or 4-hourly intervals thereafter. Doses may alternatively be given once a week, every fortnight or once a month, or more frequently, for example, two or three times weekly. Known procedures, such as those conventionally employed by the pharmaceutical industry (for example, in vivo experimentation, clinical trials, etc.), may be used to form specific formulations of the derivatives and compositions according to the invention and precise therapeutic regimes (such as daily doses of the agents and the frequency of administration).

Many studies have demonstrated key effects of IL-22 in multiple epithelial injury models in especially lung, liver, intestine, kidney, skin, pancreas and thymus. Mechanistically, several pathways within e.g. anti-apoptosis, proliferation, innate immunity, anti-oxidative stress, anti-fibrosis, and stem cell/progenitor cell recruitment have been well documented to meditate IL-22 effects in studies by multiple investigators. Key mechanistic findings have been further confirmed in vitro using human cell lines or in human ex vivo models (e.g. primary human intestinal organoids). The strong role of IL-22 in preventing cell death, securing regeneration, and controlling inflammation in epithelial injury is therefore well established.

Many studies are performed by analysing genetic models (IL-22 knock-out or transgenic overexpression) subjected to injury. In these studies, the lack of IL-22 or overexpression of IL-22 will be there at the time of injury. In other studies, IL-22 is neutralised with antibodies at the time of injury and, in some cases, IL-22 is neutralised beyond the acute injury phase (e.g. sub-acutely or well into a regeneration phase). Other studies move closer to a treatment scenario by looking at effects of exogenously administered IL-22. Important to note, when looking holistically at the available literature, is that the different models, whether knock-out, overexpression, IL-22 neutralisation before or after injury or exogenous protein dosing, paint the same picture of IL-22 protecting the injured organs and driving regeneration. This indicates a broad application and a broad time window for IL-22 treatment potential, and also shows why a longer-acting IL-22 protein than hIL-22 is required.

Thus, according to a fifth aspect of the invention, there is provided a derivative of the first aspect or a pharmaceutical composition of the third aspect, for use in a method of therapy, said method comprising administering a daily dose of between 0.001 μg/kg of body weight and 10 mg/kg of body weight of the derivative. The administration of such a daily dose is described further herein.

According to a sixth aspect of the invention, there is provided a derivative of the first aspect, or a pharmaceutical composition of the third aspect, for use in a method of treating a metabolic, liver, pulmonary, gut, kidney, CNS or skin disease, disorder or condition. Any of the different derivatives of IL-22 described or envisaged herein are expressly included in this aspect of the invention.

The metabolic disease, disorder or condition may be obesity, diabetes type 1, diabetes type 2, hyperlipidemia, hyperglycemia or hyperinsulinemia.

The liver disease, disorder or condition may be NAFLD, NASH, cirrhosis, alcoholic hepatitis, acute liver failure, chronic liver failure, ACLF, acetaminophen induced liver toxicity, acute liver injury, sclerosing cholangitis, biliary cirrhosis or a pathological condition caused by surgery or transplantation.

The pulmonary disease, disorder or condition may be COPD, cystic fibrosis, bronchiectasis, idiopathic pulmonary fibrosis, acute respiratory distress syndrome, a chemical injury, a viral infection, a bacterial infection or a fungal infection.

The gut disease, disorder or condition may be IBD, ulcerative colitis, Crohn's disease, GvHD, a chemical injury, a viral infection or a bacterial infection.

The kidney disease, disorder or condition may be acute kidney disease or chronic kidney disease.

The CNS disease, disorder or condition may be multiple sclerosis.

The skin disease, disorder or condition may be a wound, inflammatory disease or GvHD.

A method of treating a subject having a condition responsive to IL-22 treatment, such as one or more of the above diseases, disorders or conditions, with a derivative of IL-22, or a pharmaceutical composition comprising the same, is also provided.

The derivative of IL-22 has all of the features specified for the first aspect of the invention. The pharmaceutical composition has all of the features specified for the third aspect of the invention. The method of treating a subject having a condition responsive to IL-22 treatment, such as one or more of the above diseases, disorders or conditions, has all of the features specified for the fourth aspect of the invention.

There is no restriction on which derivative of IL-22 or composition as described herein should be administered to which patient. Rather, it is intended that any of the derivatives and compositions described herein can be administered to any patient as described herein.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which is not intended to limit the invention in any way.

### EXAMPLES

The materials and methods employed in the studies described in the Examples were as follows, unless where otherwise indicated.

### Derivatives & Comparators

Table 4 provides an overview of the derivatives of IL-22 and comparators represented in the data sets.

Comparators 1-13 and 19 had various backbones, types of fatty acid, linkers and sites of covalent attachment. In many cases, the linker was attached to residue 1C, as per Derivatives 1 and 2. Whilst Comparator 10 exemplifies covalent attachment at 1C, however, it lacks the G-P-G N-terminal peptide present in Derivative 1 and all of the other comparators that have a fatty acid covalently attached at 1C.

Further comparators were hIL-22, hFc-hIL-22 (a recombinant fusion protein) and Comparators 14-18, which were hIL-22 variants having one or more backbone variations only.

TABLE 4
Overview of derivatives and comparators represented in data sets

| | SEQ ID NO. (for | Backbone | Fatty acid or | Covalent | |
|---|---|---|---|---|---|
| ID | IL-22 protein) | variation | other protractor | attachment site | Linker |
| hIL-22 | SEQ ID NO. 1 | None | None | N/A | N/A |
| hFc-hIL-22 | SEQ ID NO. 1 | None | Fc | N/A | N/A |
| Derivative 1 | SEQ ID NO. 20 | G-P-G N- | C16 monoacid | 1C | γGlu-OEG- |
| | | terminal peptide, | | | OEG-εLys- |
| | | A1C | | | αAc |
| Derivative 2 | SEQ ID NO. 34 | A1C | C16 monoacid | 1C | γGlu-OEG- |
| | | | | | OEG-εLys- |
| | | | | | αAc |
| Comparator 1 | SEQ ID NO. 28 | N35Q, N64Q, | C18 diacid | 106C | γGlu-OEG- |
| | | L106C | | | OEG-C₂DA-Ac |
| | | substitutions | | | |
| Comparator 2 | SEQ ID NO. 29 | N35Q, N64Q, | C18 diacid | 95C | γGlu-OEG- |
| | | R95C | | | OEG-C₂DA-Ac |
| | | substitutions | | | |
| Comparator 3 | SEQ ID NO. 33 | L106C | C18 diacid | 106C | γGlu-OEG- |
| | | substitution | | | OEG-C₂DA-Ac |
| Comparator 4 | SEQ ID NO. 20 | G-P-G N- | C18 diacid | 1C | γGlu-OEG- |
| | | terminal peptide, | | | OEG-C₂DA-Ac |
| | | A1C | | | |
| Comparator 5 | SEQ ID NO. 21 | G-P-G-S-G-S- | C18 diacid | −7C | yGlu-OEG- |
| | | G-S-C-G-S-G- | | | OEG-C₂DA-Ac |
| | | S-G-S N- | | | |
| | | terminal peptide | | | |
| Comparator 6 | SEQ ID NO. 20 | G-P-G N- | C16 diacid | 1C | γGlu-OEG- |
| | | terminal peptide, | | | OEG-C₂DA-Ac |
| | | A1C | | | |
| Comparator 7 | SEQ ID NO. 21 | G-P-G-S-G-S- | C16 diacid | −7C | γGlu-OEG- |
| | | G-S-C-G-S-G- | | | OEG-C₂DA-Ac |
| | | S-G-S N- | | | |
| | | terminal peptide | | | |
| Comparator 8 | SEQ ID NO. 20 | G-P-G N- | C14 diacid | 1C | γGlu-γGlu- |
| | | terminal peptide, | | | γGlu-γGlu- |
| | | A1C | | | OEG-OEG- |
| | | | | | εLys-αAc |
| Comparator 9 | SEQ ID NO. 22 | G-P-G N- | C18 diacid | 1C | γGlu-OEG- |
| | | terminal peptide, | | | OEG-C₂DA-Ac |
| | | A1C, N35Q, | | | |
| | | N64Q | | | |
| Comparator 10 | SEQ ID NO. 23 | A1C, N35Q, | C18 diacid | 1C | γGlu-OEG- |
| | | N64Q | | | OEG-C₂DA-Ac |
| Comparator 11 | SEQ ID NO. 24 | G N-terminal | C18 diacid | 6C | γGlu-OEG- |
| | | peptide, H6C, | | | OEG-C₂DA-Ac |
| | | N35Q, N64Q | | | |
| Comparator 12 | SEQ ID NO. 25 | A33C, N35Q, | C18 diacid | 33C | γGlu-OEG- |
| | | N64Q | | | OEG-C₂DA-Ac |
| Comparator 13 | SEQ ID NO. 22 | G-P-G N- | C16 diacid | 1C | γGlu-OEG- |
| | | terminal peptide, | | | OEG-C₂DA-Ac |
| | | A1C, N35Q, | | | |
| | | N64Q | | | |
| Comparator 14 | SEQ ID NO. 30 | A1G | None | N/A | N/A |
| Comparator 15 | SEQ ID NO. 31 | A1G, N21D, | None | N/A | N/A |
| | | N35D, N64D | | | |
| Comparator 16 | SEQ ID NO. 32 | A1G, N35Q, | None | N/A | N/A |
| | | N64Q | | | |
| Comparator 17 | SEQ ID NO. 20 | G-P-G N- | None | N/A | N/A |
| | | terminal peptide, | | | |
| | | A1C | | | |
| Comparator 18 | SEQ ID NO. 22 | G-P-G N- | None | N/A | N/A |
| | | terminal peptide, | | | |
| | | A1C, N35Q, | | | |
| | | N64Q | | | |
| Comparator 19 | SEQ ID NO. 35 | R95C | C18 diacid | 95C | γGlu-OEG- |
| | | | | | OEG-C₂DA-Ac |

The following are exemplified protocols, merely intended to illustrate the claimed invention.

### Example 1—In Vitro Potency Study of Derivatives Comprising Fatty Monoacids

### Methods

A reporter gene assay was employed to study potency in BHK cells, which had been triple transfected with IL-22Ra, IL-10Rb and a luciferase with STAT3-induced promoter. This is a highly sensitive, high-throughput assay, which measured IL-22 receptor-mediated STAT3 activation.

A stable reporter BHK cell line was generated using the following plasmids: (i) hIL-10Rb in pcDNA3,1hygro(+), (ii) IL22R in pcDNA3,1(Zeocin) and (iii) 2×KZdel2 in pGL4.20. The cell line hence expressed the human IL-10Rb, human IL-22Ra and luciferase reporter under control of a pSTAT3 driven promoter.

On Day 0 of the assay protocol, the cells were seeded in basal media (for 500 ml: DMEM+Glutamax (Gibco, cat. no.: 31966-021), 10% (w/v) fetal calf serum (FCS; contains albumin) (50 ml) and 1% (w/v) penicillin-streptomycin (P/S) (5 ml)) at 15,000-20,000 cells/well in a 96-well plate (Corning #3842, black, clear bottom). On Day 1, the media was removed by inverting the plate. Fresh basal media was added, at 50 μl per well, and the cells were incubated for 60 minutes.

The derivative of IL-22 was tested alongside hIL-22 as a comparator in duplicate.

Thus, 50 μl of a diluted derivative or comparator (diluted in basal media) was added to each well and the plate left for four hours. The derivative and comparator were therefore 2× diluted, as they were diluted into the 50 μl media already in the wells. The stimulation was ended after four hours by adding 100 μl Steadylite plus reagent (Perkin Elmer cat no. 6066759). The plate was sealed with TopSeal A, shaken at 450 rpm for 15 minutes, then read using Mithras or a similar system no later than after 12 hours.

Data analysis was performed using Graphpad Prism. The half-maximal effective concentration (EC₅₀) of the derivative or comparator was assessed as a measure of its potency. EC₅₀ was determined using Log(compound) vs response—variable slope (4p). Hill slope was constrained to 1 as standard.

### Results

Table 5 shows the EC₅₀ of the derivative and comparator measured in the BHK cell reporter gene assay for IL-22 receptor-mediated STAT3 activation. “n” depicts the number of individual assay runs. All assay runs were carried out under the same conditions, but separate individual experiments were performed on separate days.

TABLE 5
EC₅₀ values for derivative and comparator in BHK cell assay

| ID | EC₅₀ (nM) | n = |
|---|---|---|
| hIL-22 | 0.07 | 13 |
| Derivative 1 | 0.06 | 2 |

As the BHK cell assay contained high amounts of albumin, the measured EC₅₀ incorporated the effect of albumin binding when testing the derivative.

Derivative 1, having backbone variation and a covalently attached C16 monoacid, was shown to be equipotent to hIL-22

### Conclusion

A surprising equipotency was observed of Derivative 1 and hIL-22. In comparison, and as aforementioned, Genentech reports a 34-fold reduction in in vitro potency for its Fc fusion of IL-22.

Thus, derivatives of IL-22 maintain high potency in the presence of albumin. Cys substitution, an N-terminal tripeptide extension and fatty monoacid covalent attachment are clearly well-tolerated.

The data hence illustrate that the derivatives of the invention demonstrate good bioavailability and potency, so offering a new and improved treatment for a diverse range of indications, including metabolic, liver, pulmonary, gut, kidney, CNS and skin diseases, disorders and conditions.

While certain features of the invention have been illustrated and described herein, many modifications and equivalents will occur to those of ordinary skill in the art. It is, therefore, to be understood that the claims are intended to cover all such modifications and equivalents as fall within the true spirit of the invention.

### Example 2—Mean Residence Time and Potency Study of Derivatives Comprising Fatty Monoacids and Comparators Comprising Fatty Diacids

### Methods

In the following, two methods are described. The first relates to pharmacokinetic studies carried out on Derivative 1 and Comparators 14, 8, 6 and 4 in rats to determine mean residence time (MRT). The second relates to a potency assay to determine the in vitro EC₅₀ (nM) for the same derivative and comparators.

### Measuring In Vivo MRT in Rats

Four rats were dosed with a derivative or comparator and three blood samples were taken at each of the following time points: 5 min, 15 min, 30 min, 45 min, 60 min, 75 min, 90 min, 105 min, 120 min, 150 min, 3 h, 4 h, 6 h, 8 h and 24 h. Each rat had 17 samples taken during the course of the study. After the last sample was taken, the rats were euthanised by carbon dioxide.

Blood samples (100 μl) were taken from the rats by tongue blood and transferred to EDTA tubes (Microvette® VetMed 200 K3E, Sarstedt nr 09.1293.100). The blood was centrifuged for five minutes at 8000 G, 4° ° C. within 20 minutes of being drawn. The plasma samples (40-50 μl) were transferred to half micronic tubes.

Plasma levels of derivative or comparator were measured using in-house developed luminescent oxygen channeling (LOCI®) assays as previously described (Poulsen et al. J Biomol Screen, 2007, 12(2):240-7). During the assays, a concentration-dependent bead-analyte-immune complex was created, resulting in light output, which was measured on a Perkin Elmer Envision reader. Coupling of antibodies to beads, biotinylation of antibodies and LOCI® assay procedure were performed as previously described (Petersen et al., J Pharmaceut Biomed, 2010, 51(1):217-24). Calibrators and quality control (QC) samples were produced in the same matrix as the study samples. Assay precision (% CV) was assessed and shown to be lower than 20% for all the tested samples.

The assay used anti-human IL-22 monoclonal antibody (R&D Systems MAB7822)-conjugated acceptor beads together with biotinylated monoclonal antibody (R&D Systems BAM7821; raised against human IL-22) and generic streptavidin-coated donor beads. The lower limit of quantification (LLOQ) for human IL-22 in rat plasma was 4 pM. Each derivative or comparator was, however, measured against a calibrator row of the same derivative or comparator. The cross-reactivity of each derivative or comparator against hIL-22 was measured and used to adjust the assay sensitivity.

Pharmacokinetic parameters including MRT (i.e. the time that the drug spends in the body prior to elimination once absorption has been completed), were calculated using Phoenix WinNonlin Professional 6.4 (Pharsight Inc).

### Measuring In Vitro EC₅₀ (nM) in BHK Cells

Reporter gene assay in BHK cells, which had been triple transfected with IL-22Ra, IL-10Rb and a luciferase with STAT3-induced promoter were used. This is a highly sensitive, high-throughput assay, which measured IL-22 receptor-mediated STAT3 activation.

A stable reporter BHK cell line was generated using the following plasmids: (i) hIL-10Rb in pcDNA3,1hygro(+), (ii) IL22R in pcDNA3,1(Zeocin) and (iii) 2×KZdel2 in pGL4.20. The cell line hence expressed the human IL-10Rb, human IL-22Ra and luciferase reporter under control of a pSTAT3 driven promoter.

On Day 0 of the assay protocol, the cells were seeded in basal media (for 500 ml: DMEM+Glutamax (Gibco, cat. no.: 31966-021), 10% (w/v) fetal calf serum (FCS; contains albumin) (50 ml) and 1% (w/v) penicillin-streptomycin (P/S) (5 ml)) at 15,000-20,000 cells/well in a 96-well plate (Corning #3842, black, clear bottom). On Day 1, the media was removed by inverting the plate. Fresh basal media was added, at 50 μl per well, and the cells were incubated for 60 minutes.

Derivative 1 and Comparators 14, 8, 6 and 4 were tested. The “n” number of assay runs ranged from 1-24.

Thus, 50 μl of a derivative or comparator (diluted in basal media) was added to each well and the plate left for four hours. The derivative and comparators were therefore 2× diluted, as they were diluted into the 50 μl media already in the wells. The stimulation was ended after four hours by adding 100 μl Steadylite plus reagent (Perkin Elmer cat no. 6066759). The plate was sealed with TopSeal A, shaken at 450 rpm for 15 minutes, then read using Mithras or a similar system no later than after 12 hours.

Data analysis was performed using Graphpad Prism. The half maximal effective concentration (EC₅₀) of each derivative or comparator was assessed as a measure of its potency. EC₅₀ was determined using Log(compound) vs response—variable slope (4p). Hill slope was constrained to 1 as standard.

### Results

The mean residence time in rats and the in vitro EC₅₀ of Derivative 1 and the tested comparators is shown in Table 6. “n” depicts the number of individual assay runs. All assay runs were carried out under the same conditions, but separate individual experiments were performed on separate days.

TABLE 6
Mean residence time in rats and in vitro
EC₅₀ for Derivative 1 and comparators

| | Fatty acid | Mean | |
|---|---|---|---|
| | or other | Residence | |
| ID | protractor | Time (h) | In vitro EC₅₀ (nM) |
| Comparator 14 | None | 0.18 | 0.06 (n = 8) |
| Derivative 1 | C16 monoacid | 0.8 | 0.06 (n = 2) |
| Comparator 8 | C14 diacid | 0.6 | NA |
| Comparator 6 | C16 diacid | 1.4 | 0.30 (n = 6) |
| Comparator 4 | C18 diacid | 5.7 | 0.48 (n = 24) |

The data show that the mean residence time increased relative to hIL-22 when the fatty acid attached to the same backbone was changed from monoacid to diacid (compare the data for Derivative 1 and Comparator 6). Increasing the length of the diacid also increased the mean residence time (compare the data for Comparators 8, 6 and 4).

Data also showed that the monoacid attachment, although increasing the mean residence time compared to no attachment, did not negatively impact the potency (compare data for Derivative 1 and Comparator 14), in distinct contrast to the diacid attachments (compare data for Derivative 1 and Comparator 6).

### Conclusion

Using mean residence time measured in rat, this comparison study confirmed that the impact of increasing fatty diacid chain length was an increased half-life with an acceptable reduction in potency compared to wild type human IL-22. Similar results were also shown in mini-pig studies (see Example 3). The half-life as measured in mini-pigs (Example 3) showed that C18 fatty diacids have a similar prolonged half-life to Fc-fusions used as protractors for IL-22, whereas C16 fatty diacids have a shorter half-life than both and all are considerably longer than the wild-type human IL-22.

In the current data set in rats, we have shown that attachments of fatty monoacids are indeed resulting in a shorter mean residence time compared to fatty diacids of the same length (comparing Derivative 1 and Comparator 6). At the same time, derivatives comprising fatty monoacid lipidations have been shown to maintain the same half-life as the IL-22 wild-type like Comparator 14, yet increase the mean residence time by at least four times compared to the same.

In the in vivo examples presented herein, we have also shown that the balance between the potency reduction and the increased half-life still leads to a biological effect and makes the derivatives of this invention suitable candidates for drug development and treatment of disease.

These comparison data between fatty monoacid and fatty diacid attachments and length bring us to the conclusion that all tested fatty acid attachments are indeed suitable for different drug development purposes, depending on the desired administration or diseases or conditions to be treated. Thus, an optimum use of fatty monoacids as protracting means for IL-22 proteins may be situations in which a protracted action is desired, but with a shorter circulation time than for diacids and Fc-fusions.

### Example 3—Pharmacokinetic Study of Comparators Comprising Fatty Diacids

### Methods

Pharmacokinetic studies were carried out on selected comparators in mice (n=27), rats (n=4-8) and minipigs (n=2-5) including hIL-22 and hFc-hIL-22.

### (i) Mice & Rats

30 8-week old C57Bl/6 male mice and five Sprague Dawley male rats were obtained from Taconic Biosciences. The mice were housed in groups of 10. Animals were acclimatised for one week prior to the experiments. Body weight was measured prior to dosing, which is important for pharmacokinetic calculations. The animals were awake throughout the experiment, with access to food and water.

All comparators were prepared as 0.3 mg/ml solutions in PBS, pH 7.4, for use in mice and 0.5 mg/ml solutions for use in rats. A dose of 2.0 mg/kg was tested in mice. A dose of 1 mg/kg was tested in rats.

The comparators were administered to the animals subcutaneously. Blood samples were taken at specific time points after dosing.

Sparse sampling was used in mice; thus, 27 mice were dosed with a comparator and blood samples were taken from three different mice at each of the following time points: 5 min, 15 min, 30 min, 45 min, 60 min, 75 min, 90 min, 105 min, 120 min, 150 min, 3 h, 4 h, 6 h, 8 h, 16 h, 24 h, 32 h and 48 h. Each mouse therefore had just two samples taken during the course of the study. After the last sample was taken, the mice were euthanised by cervical dislocation.

Five rats were dosed with a comparator and three blood samples were taken at each of the following time points: 5 min, 15 min, 30 min, 45 min, 60 min, 75 min, 90 min, 105 min, 120 min, 150 min, 3 h, 4 h, 6 h, 8 h and 24 h. Each rat had 17 samples taken during the course of the study. After the last sample was taken, the rats were euthanised by carbon dioxide.

Blood samples (100 μl) were taken from mice and rats by tongue blood and transferred to EDTA tubes (Microvette® VetMed 200 K3E, Sarstedt nr 09.1293.100). The blood was centrifuged for five minutes at 8000 G, 4° ° C. within 20 minutes of being drawn. The plasma samples (40-50 μl) were transferred to half micronic tubes.

### (ii) Minipigs

9-month old female Göttingen minipigs having a body weight of approximately 15 kg were obtained from Ellegaard Göttingen Minipigs A/S. An acclimatisation period of approximately 18 days was allowed before surgery (insertion of catheters), during which time the minipigs were socialised and trained for subcutaneous dosing and blood sampling from the catheter. Three to five days before surgery the minipigs were single-housed. Six days before dosing all minipigs had two central venous catheters (Cook Medical, C-TPNS-6.5-90-REDO, silicon, size 6.5 french, 106 cm long type TPN) inserted, which allowed a recovery time after surgery of at least five days before study start (dosing).

All comparators were prepared as solutions in PBS, pH 7.4. The doses used were 0.1 mg/kg (administered intravenously) or 0.2 mg/kg (administered subcutaneously).

Minipigs were lightly anaesthetized with Propofol during the dosing. Intravenous injections were administered to minipigs through the long central catheter. After administration, the catheter was flushed with 10 ml sterile saline. Subcutaneous injection was given in 5 mm depth using a 25 G needle. The needle was kept in the skin for 10 s after injection to avoid back flow.

Blood samples were taken from the minipigs at the following time points after intravenous dosing: 1.5 h, 2 h, 3 h, 4 h, 6 h, 8 h, 10 h, 12 h, 24 h, 28 h, 48 h, 72 h, 96 h, 144 h, 168 h, 192 h, 216 h, 240 h, 264 h, 312 h, 336 h, 360 h, 384 h, 408 h, 432 h and 480 h. Blood samples were taken at the following time points after subcutaneous dosing: 1.5 h, 2 h, 3 h, 4 h, 5 h, 6 h, 8 h, 10 h, 12 h, 14 h, 16 h, 18 h, 20 h, 22 h, 24 h, 26 h, 28 h, 46 h, 52 h, 72 h, 96 h, 144 h, 168 h, 192 h, 216 h, 240 h, 264 h, 312 h, 336 h, 360 h, 384 h, 408 h, 432 h and 480 h.

Blood samples (1 ml) were collected from minipigs in EDTA tubes (1.3 ml tube containing K3EDTA to yield 1.6 mg K3EDTA/ml blood (Sarstedt, Germany)). Samples were kept on wet ice for a maximum of 30 min until centrifugation (10 min, 4° C., 2000 G). 200 μl plasma was transferred into Micronic tubes for measurement of the comparators and stored at −20° C. until analysis.

(iii) Sample Processing

Plasma levels of comparators were measured using in-house developed luminescent oxygen channeling (LOCIR) assays as previously described (Poulsen et al. J Biomol Screen, 2007, 12(2):240-7). During the assays, a concentration-dependent bead-analyte-immune complex was created, resulting in light output, which was measured on a Perkin Elmer Envision reader. Coupling of antibodies to beads, biotinylation of antibodies and LOCI assay procedure were performed as previously described (Petersen et al., J Pharmaceut Biomed, 2010, 51(1):217-24). Calibrators and quality control (QC) samples were produced in the same matrix as the study samples. Assay precision (% CV) was assessed and shown to be lower than 20% for all the tested samples.

The assay used anti-human IL-22 monoclonal antibody (R&D Systems MAB7822)-conjugated acceptor beads together with biotinylated monoclonal antibody (R&D Systems BAM7821; raised against human IL-22) and generic streptavidin-coated donor beads. The lower limit of quantification (LLOQ) for human IL-22 in rat plasma was 4 pM. Each comparator was, however, measured against a calibrator row of the same comparator. The cross-reactivity of each comparator against hIL-22 was measured and used to adjust the assay sensitivity.

Plasma concentration-time profiles were measured for minipigs using a non-compartmental analysis (NCA) in Phoenix WinNonlin Professional 6.4 (Pharsight Inc). Calculations were performed using individual concentrations, weighting by 1/(Y*Y), and using linear log trapezoidal. Intravenous dosing was used because circulatory elimination half-life (T_{1/2}) was the primary screening parameter. Clearance and volume of distribution were secondary parameters of interest, hence the reason for frequent blood samples during day 1 of the study.

The sole parameter measured to assess pharmacokinetics in mice and rats was circulatory elimination half-life (T_{1/2}). In minipigs, the additional parameters measured were maximum (peak) plasma concentration following drug administration (Cₘₐₓ), time to reach Cₘₐₓ (Tₘₐₓ), area under the plasma drug concentration-time curve (AUC; which reflects the actual body exposure to drug after administration of a dose of the drug) normalised for drug dose (AUC/D), mean residence time (MRT; i.e. the time that the drug spends in the body prior to elimination once absorption has been completed), mean absorption time (MAT) and systemic availability of the administrated dose (i.e. bioavailability; F). MAT is calculated as MRT following subcutaneous administration (MRT_{SC}) minus MRT following intravenous administration (MRT_{IV}).

### Results

Table 7 shows the results obtained in mice, Table 8 shows the results obtained in rats and Tables 9 and 10 show the results obtained in minipigs. ND=not determined. IV=intravenous administration. SC=subcutaneous administration.

TABLE 7
Pharmacokinetic data obtained in mice

| ID | Route of Administration | T_{1/2} (h) |
|---|---|---|
| hFC-hIL-22 | IV | ND |
| | SC | 30.0 |
| Comparator 14 | IV | 0.4 |
| | SC | 1.4 |
| Comparator 16 | IV | 0.4 |
| | SC | 0.7 |
| Comparator 4 | IV | 6.0 |
| | SC | 8.3 |
| Comparator 9 | IV | 6.6 |
| | SC | 9.1 |

As shown in Table 7, hIL-22 variants having backbone variations only (Comparators 14 and 16) had a short circulatory half-life, regardless of the route of administration. Protraction with Fc fusion (hFc-hIL-22) considerably increased half-life. Covalent attachment of fatty acid (C18 diacid; Comparators 4 and 9) resulted in an intermediary circulatory half-life in mice. The comparators circulated for longer in mice when administered subcutaneously compared to intravenously.

TABLE 8
Pharmacokinetic data obtained in rats

| ID | Route of Administration | T_{1/2} (h) |
|---|---|---|
| Comparator 14 | IV | 0.2 |
| | SC | ND |
| Comparator 4 | IV | 11.1 |
| | SC | 10.9 |
| Comparator 6 | IV | 4.3 |
| | SC | 9.7 |
| Comparator 9 | IV | 10.8 |
| | SC | 12.3 |

As shown in Table 8, the hIL-22 variant having a backbone variation only (Comparator 14) had a short circulatory half-life. Covalent attachment of fatty acid (Comparators 4, 6 and 9) resulted in an increased circulatory half-life in rats, regardless of the fatty acid (C16 vs C18 diacid) employed and route of administration. The comparators typically circulated for longer when administered subcutaneously compared to intravenously.

Similar results are expected for the derivatives of the invention comprising fatty monoacids. Indeed, from the above data it is evident that the covalent attachment of a C16 or C18 fatty acid presents a relatively long half-life for the longer fatty acid chain compared to the shorter one in rat, and that the circulation time is extended when administered subcutaneously compared to intravenously. The same tendencies can be expected with monoacids, albeit with generally shorter half-lives compared to fatty diacids.

TABLE 9
Pharmacokinetic data obtained in minipigs

| ID | Administration route | T_{1/2} (h) |
|---|---|---|
| hIL-22 | IV | 4.6 |
| | SC | 6.6 |
| hFc-hIL-22 | IV | 65.4 |
| | SC | 141.0 |
| Comparator 14 | IV | 3.9 |
| | SC | 8.7 |
| Comparator 15 | IV | 3.5 |
| | SC | 3.7 |
| Comparator 4 | IV | 53.9 |
| | SC | ND |
| Comparator 9 | IV | 66.5 |
| | SC | 106.0 |
| Comparator 13 | IV | ND |
| | SC | 40.0 |

As shown in Table 9, the hIL-22 variants having backbone variations only (Comparators 14 and 15) had a short circulatory half-life, comparable with hIL-22. The comparator Fc fusion (hFc-hIL-22) and all of the other comparators (Comparators 4, 9 and 13) had a significantly increased circulating half-life. Comparators 4, 9 and 13 had a circulating half-life of over 50 hours in minipig when administered intravenously, which was on par with the comparator IL-22-Fc fusion.

TABLE 10
Pharmacokinetic data obtained in minipigs

| | Admin- | | | AUC/D | | | |
|---|---|---|---|---|---|---|---|
| | istration | Cmax | Tmax | (h*kg*pmol/ | MRT | MAT | F |
| ID | route | (nmol/l) | (h) | l/pmol) | (h) | (h) | (%) |
| hFc- | IV | 56.0 | 0.05 | 407 | 91.7 | ND | ND |
| hIL-22 | SC | 14.8 | 5.00 | 365 | 192.0 | 100 | 89.7 |
| Compar- | IV | 98.9 | 0.05 | 551 | 85.6 | ND | ND |
| ator 9 | SC | 37.6 | 8.00 | 399 | 146 | 60.3 | 72.4 |

As shown in Table 10, a faster MAT was demonstrated for the Comparator 9 compared to the comparator Fc fusion (hFc-IL-22). MAT is a more precise measure of drug uptake than simply comparing Tₘₐₓ, as it also takes into consideration differences in Cₘₐₓ (Tₘₐₓ is influenced by both dose and Cₘₐₓ). Minipigs were used for this study, rather than mice or rats, because of their similarity to humans.

### Conclusion

The known fatty acid alkylated GLP-1 derivative, semaglutide, has a half-life of 46 hours in minipig (Lau et al., J Med Chem, 2015, 58(18): 7370-80) and a half-life of 160 hours in man, corresponding to a once-weekly dosing profile with a peak to trough ratio of 2. The half-life of the Fc-fusion GLP-1 derivative, dulaglutide, is similar.

The half-life demonstrated by Comparators 4, 9 and 13 in minipig, of at least 40 hours when administered subcutaneously and over 50 hours when administered intravenously, is therefore assumed to correspond to a once-weekly dosing profile in man with a peak to trough ratio of 2.

The data hence show that the comparators enhanced circulating half-lives of IL-22 and demonstrated optimised pharmacokinetic and pharmacodynamic properties, so offering a new and improved treatment for a diverse range of indications, including metabolic, liver, pulmonary, gut, kidney, eye, thymus, pancreas, and skin diseases, disorders and conditions.

Similar results are expected for the derivatives of the invention comprising fatty monoacids. Indeed, from the above data set and the previous and following Examples regarding potency, half-life and mean residence time of IL-22 variants as disclosed herein, it has been made plausible that a similar biologic effect for fatty monoacid attachments as compared to IL-22 proteins comprising fatty diacid attachments, albeit with generally shorter half-lives and higher potency compared to the fatty diacid results presented here, can be expected. This may be particularly desirable in situations in which a protracted action is desired, but with shorter circulation time than for diacids and Fc-fusion.

### Example 4—In Vitro Potency Study of Comparators Comprising Fatty Diacids

### Methods

Two in vitro assays were employed to study potency of IL-22 proteins with modifications compared to SEQ ID NO. 1.

The first was a reporter gene assay in BHK cells, which had been triple transfected with IL-22Ra, IL-10Rb and a luciferase with STAT3-induced promoter. This is a highly sensitive, high-throughput assay, which measured IL-22 receptor-mediated STAT3 activation.

A stable reporter BHK cell line was generated using the following plasmids: (i) hIL-10Rb in pcDNA3,1hygro(+), (ii) IL22R in pcDNA3,1(Zeocin) and (iii) 2×KZdel2 in pGL4.20. The cell line hence expressed the human IL-10Rb, human IL-22Ra and luciferase reporter under control of a pSTAT3 driven promoter.

On Day 0 of the assay protocol, the cells were seeded in basal media (for 500 ml: DMEM+Glutamax (Gibco, cat. no.: 31966-021), 10% (w/v) fetal calf serum (FCS; contains albumin) (50 ml) and 1% (w/v) penicillin-streptomycin (P/S) (5 ml)) at 15,000-20,000 cells/well in a 96-well plate (Corning #3842, black, clear bottom). On Day 1, the media was removed by inverting the plate. Fresh basal media was added, at 50 μl per well, and the cells were incubated for 60 minutes.

Comparators 4, 6, 7 and 9-13 were tested alongside hIL-22 and hIL-22 variants having backbone variations only as comparators. The “n” number of assay runs ranged from 1-24.

Thus, 50 μl of a comparator (diluted in basal media) was added to each well and the plate left for four hours. The comparators were therefore 2× diluted, as they were diluted into the 50 μl media already in the wells. The stimulation was ended after four hours by adding 100 μl Steadylite plus reagent (Perkin Elmer cat no. 6066759). The plate was sealed with TopSeal A, shaken at 450 rpm for 15 minutes, then read using Mithras or a similar system no later than after 12 hours.

Data analysis was performed using Graphpad Prism. The half maximal effective concentration (EC₅₀) of each comparator was assessed as a measure of its potency. EC₅₀ was determined using Log(compound) vs response—variable slope (4p). Hill slope was constrained to 1 as standard.

The second in vitro potency assay measured pSTAT3 in HepG2 cells—a human liver-derived cell line endogenously expressing IL-22Ra and IL-10Rb.

On Day 1, HepG2 Cells were plated at 25,000-30,000 cells/well in a 96-well plate (Biocoat #35-4407 Becton Dickinson). The cell media used for plating and passaging was DMEM(1×)+25 mM (4.5 g/l) glucose, −pyruvate (Gibco, cat. no. 61965-026)+10% (w/v) FCS+1% (w/v) P/S. On Day 2, the cells were ready for assay. The cells were starved with 0.1% (w/v) FCS (i.e. a very low albumin concentration) in DMEM (Gibco, cat. no. 61965-026) −50 μl was added to each well and left for 60 minutes.

Tests were performed in seven concentrations of each comparator as standard (0.001, 0.01, 0.1, 1, 10, 100, 1000 nM) using technical duplicates. Thus, 50 μl of a diluted comparator (diluted in 0.1% (w/v) FCS in DMEM) was added to each well and the plate left for 15 minutes. The comparators were therefore 2× diluted, as they were diluted into the 50 μl media already in the wells. To lyse the cells, media was removed from the cells and 50 μl of freshly prepared 1×lysis buffer (SureFire lysis buffer from kit) was added to each well. The plate was agitated at 350 rpm for 10 minutes at room temperature.

The AlphaScreen® SureFire® STAT3 (p-Tyr705) assay protocol (Perkin Elmer cat. no. TGRS3S (500-10K-50K)) was followed to measure IL-22 induced phosphorylation of STAT3. In this regard, 4 μl of lysate was transferred to a 384-well proxiplate for assay (adding 4 μl of positive and negative control). Immediately prior to use, Acceptor mix was prepared (by diluting Activation buffer 5-fold in reaction buffer and diluting Acceptor beads 50-fold in the diluted buffer). 5 μl of Acceptor mix was added to each well, the plate sealed with Topseal A adhesive film and incubated for two hours at room temperature. Immediately prior to use, Donor mix was prepared (by diluting Donor beads 20-fold in Dilution buffer). 2 μl of donor mix was added to the wells under subdued light. The plate was again sealed with Topseal A adhesive film and incubated for two hours at room temperature. The plate was read on an Alpha Technology-compatible plate reader.

Data analysis was performed using Graphpad Prism. First, a non-linear regression was conducted using Log(compound) vs. response—variable slope (4p) analysis in Prism. Hill slope was constrained to 1. The Y=top from the control compound (hIL-22) was then used for a normalisation in Prism. 0% was set to the smallest value in each data set and 100% to Y-top from the above non-linear regression (for the control). A non-linear regression was repeated as above and % activity/wt and EC₅₀ of the tested comparators read in the results under top and EC₅₀ respectively.

### Results

Table 11 shows the EC₅₀ of key comparators measured in the BHK cell reporter gene assay for IL-22 receptor mediated STAT3 activation.

TABLE 11
EC₅₀ values for key comparators in BHK cell assay

| ID | EC₅₀ (nM) |
|---|---|
| hIL-22 | 0.07 |
| Comparator 14 | 0.06 |
| Comparator 18 | 0.19 |
| Comparator 17 | 0.09 |
| Comparator 4 | 0.48 |
| Comparator 6 | 0.30 |
| Comparator 7 | 0.18 |
| Comparator 9 | 0.61 |
| Comparator 10 | 0.09 |
| Comparator 11 | 1.24 |
| Comparator 12 | 0.28 |
| Comparator 13 | 0.37 |

As the BHK cell assay contained high amounts of albumin, the measured EC₅₀ incorporated the effect of albumin binding when testing the comparators.

Comparator 17, an IL-22 variant having backbone variations only, was shown to be equipotent to hIL-22. Comparator 6, having the same backbone as Comparator 17, but covalently attached to a medium affinity albumin binder (C16 diacid), exhibited a four-fold reduction in potency compared to hIL-22. Comparator 4, again having the same backbone, but covalently attached to a high affinity albumin binder (C18 diacid), exhibited only a seven-fold reduction in potency compared to hIL-22.

A scan of alkylation positions and backbone variations has been performed with off-set in a 35Q, 64Q background (i.e. two of the three IL-22 glycosylation sites mutated) by comparing the results for Comparators 9-12. The results obtained with these comparators demonstrate that Cys substitution and fatty acid covalent attachment may be tolerated in several (select) positions, which was surprising to the inventors.

Similar results are expected for the derivatives of the invention comprising fatty monoacids. Indeed, from the above data set and the previous and following Examples regarding potency, half-life and mean residence time of IL-22 variants as disclosed herein, it has been made plausible that Cys substitution and fatty acid covalent attachment may be tolerated in several (select) positions for IL-22 derivatives comprising fatty monoacids, instead of fatty diacids.

Table 12 shows the EC₅₀ of key comparators measured in the HepG2 cell assay for pSTAT3.

TABLE 12
EC₅₀ values for key comparators in HepG2 cell assay

| ID | EC₅₀ (nM) |
|---|---|
| hIL-22 | 3.88 |
| Comparator 14 | 4.73 |
| Comparator 17 | 12.11 |
| Comparator 4 | 10.13 |
| Comparator 5 | 6.98 |
| Comparator 9 | 14.86 |

In the HepG2 cell assay with endogenous expression level of receptors, little signal amplification and no albumin, Comparator 4 had a 2.5-fold reduced potency compared to hIL-22 (similar to Comparator 17, an hIL-22 variant having the same backbone as Comparator 4, but no fatty acid).

Table 13 collates the results from both the BHK and HepG2 cell assays to assess fatty acid covalent attachment in N-terminal extension and mutation of glycosylation sites. ND=not determined.

TABLE 13
EC₅₀ values for key comparators
and hIL-22 in BHK and HepG2 cell

| ID | BHK cells EC₅₀ (nM) | HepG2 cells EC₅₀ (nM) |
|---|---|---|
| hIL-22 | 0.07 | 3.88 |
| Comparator 4 | 0.48 | 10.13 |
| Comparator 5 | ND | 6.98 |
| Comparator 7 | 0.18 | ND |
| Comparator 9 | 0.61 | 14.86 |

assays

Comparator 9 differs from Comparator 4 by the additional N35Q and N64Q substitutions (two out of three glycosylation sites mutated), yet they are equipotent (with a tendency to slightly lower potency for Comparator 9).

Similar results are expected for the derivatives of the invention comprising fatty monoacid attachments. Indeed, the effect of these two substitutions, N35Q and N64Q, are expected to have a similar effect in derivatives comprising fatty monoacids.

Whilst Comparators 5 and 7 have a 15-mer N-terminal extension, with the Cys residue for fatty acid attachment in the extension (−7C), this is surprisingly shown to be well-tolerated.

Again, a similar effect of N-terminal extensions applied to derivatives of the invention comprising fatty monoacid attachments may be expected. Similar results are indeed expected for derivatives comprising fatty monoacid attachments, based on the above data set and the previous and following Examples regarding potency, half-life and mean residence time of IL-22 variants as disclosed herein, albeit with generally shorter half-lives and higher potency compared to fatty diacid results.

### Conclusion

The potency reduction observed with fatty acid covalent attachment in the tested comparators was primarily driven by albumin binding, with backbone substitutions having little contribution. This was demonstrated by the surprising equipotency of Comparator 17 and hIL-22. In comparison, and as aforementioned, Genentech reports a 34-fold reduction in in vitro potency for its Fc fusion of IL-22.

In the HepG2 cell assay with very low albumin levels, Comparator 4 (the comparator that showed a seven-fold potency reduction in the BHK assay (with albumin binding)) showed only a 2.5-fold reduction in potency compared to hIL-22.

The equipotency of Comparators 4 and 9 (Table 13) showed that the 35Q and 64Q mutations are surprisingly tolerated without effect on potency.

Thus, the comparators maintain high potency in the presence of albumin and are near equipotent with hIL-22 in the absence of albumin. Cys substitution and fatty acid covalent attachment are tolerated in several positions.

The data hence show that the comparators demonstrate good bioavailability and potency, so offering a new and improved treatment for a diverse range of indications, including metabolic, liver, pulmonary, gut, kidney and skin diseases, disorders and conditions. As above, similar results are expected for the derivatives of the invention comprising fatty monoacids instead.

### Example 5—In Vivo Efficacy Study in Diabetes of Comparators Comprising Fatty Diacids

This study was designed to investigate the effect of once-daily dosing with comparators of derivatives of the invention for 8-16 days in a diabetes mouse model. The study was done in treatment (not preventive) mode, meaning that diabetes pathology was developed before dosing was initiated. As the mouse model has a fatty liver (leptin receptor knockout), it also functions as a metabolic model of liver disease.

### Methods

7-8 week old male C57BKS db/db mice were obtained from Charles River Laboratories (Day −10) and acclimatised for at least one week prior to the start of experiments. One week after arrival (Day −3), the mice were randomised and housed in groups of 10 (or singly for the food intake study). On Day −3, and on each of Days 1-16 of the study, blood glucose and food intake were measured.

Comparator 4 was tested alongside an Fc fusion of IL-22 (hFc-hIL-22) as a further comparator and vehicle only as a negative control. 1. Each agent was administered subcutaneously in a once-daily dose of 0.1, 0.25, 0.5 or 1.0 mg/kg on each of Days 1-16 days in the diabetic db/db mice (n=6-10 in each group). Food intake was reduced following comparator/control dosing.

Blood glucose was measured daily over the study duration. Eye blood samples were taken at termination in anaesthetised mice. 500 μl blood was collected into EDTA tubes. The samples were kept on ice and centrifuged for five minutes at 6000 G at 4° C. within 20 minutes. Plasma was separated into 0.75 ml micronic tubes and immediately frozen for later measurement of component concentrations.

As well as measuring comparator concentration, the plasma levels of target engagement biomarkers (the liver-derived acute phase proteins, haptoglobin and Serum Amyloid P component (SAP), and the gut-derived Peptide YY (PYY)) were measured at study end. Haptoglobin was measured on a COBAS instrument (Roche Diagnostics) with commercial kit according to the manufacturer's instructions. PYY was measured with a commercial ELISA assay (ALPCO) recognising mouse and rat PYY according to manufacturer's instructions. SAP was measured with a commercial ELISA assay (R&D Systems) recognising mouse Pentraxin 2/SAP according to manufacturer's instructions.

### Results

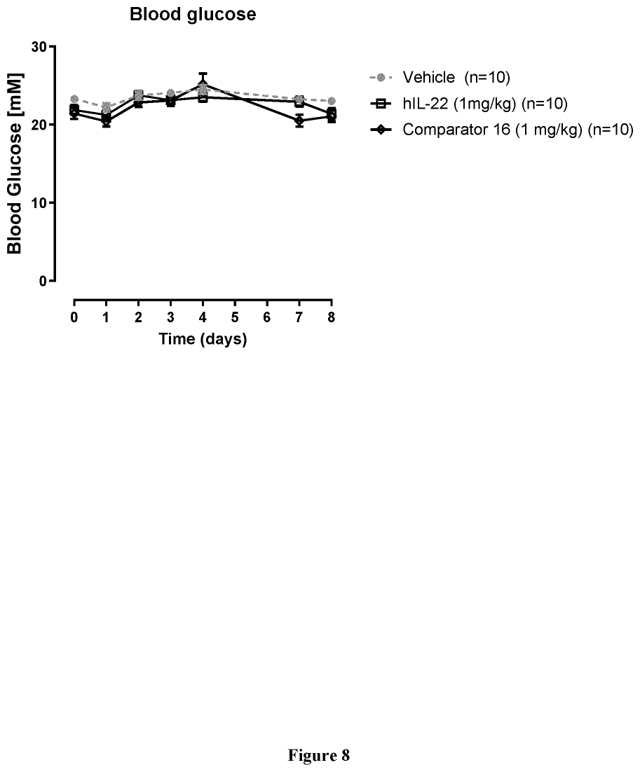

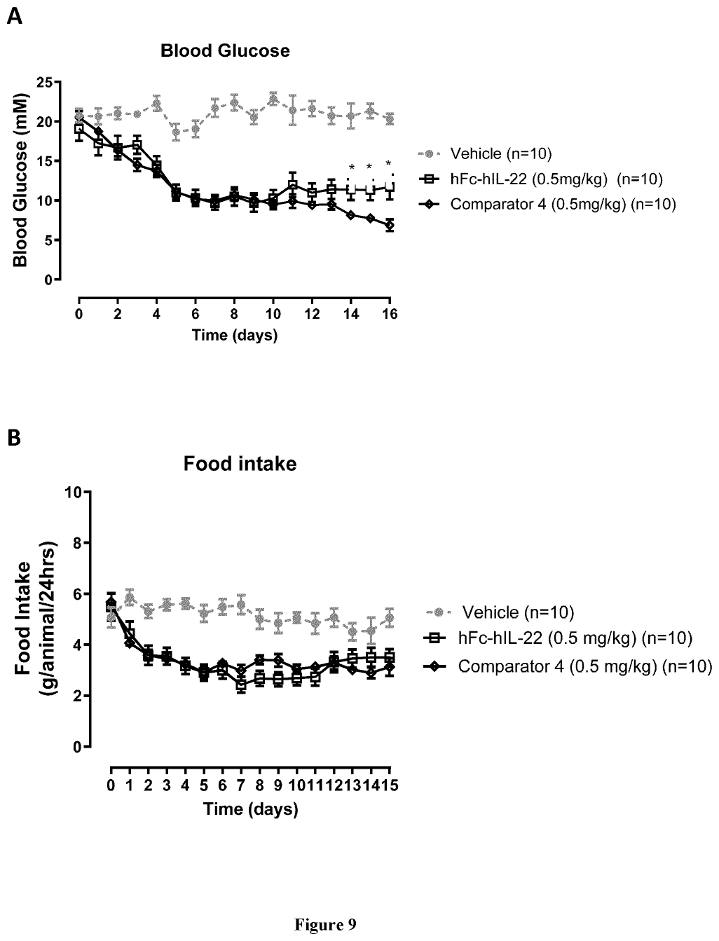

Blood glucose levels over the duration study are shown in FIGS. 8 and 9A.

As can be seen from FIG. 8, hIL-22 and an hIL-22 variant having backbone variations only (Comparator 16) failed to reduce blood glucose over the course of the study compared to the vehicle control.

As can be seen from FIG. 9A, Comparator 4 and hFc-hIL-22 both reduced blood glucose in a comparable manner toward normal levels with a slightly higher efficacy of Comparator 4 in the last days of the study, despite higher target engagement of hFc-hIL-22 reflecting a higher steady state exposure level in the specific study. A reduction in food intake was observed in the treated animals compared to the vehicle control (see FIG. 9B). Comparator 4 thus normalised blood glucose in the db/db model in a similar manner to hFc-hIL-22; as above, no such effect was observed with hIL-22 or Comparator 16.

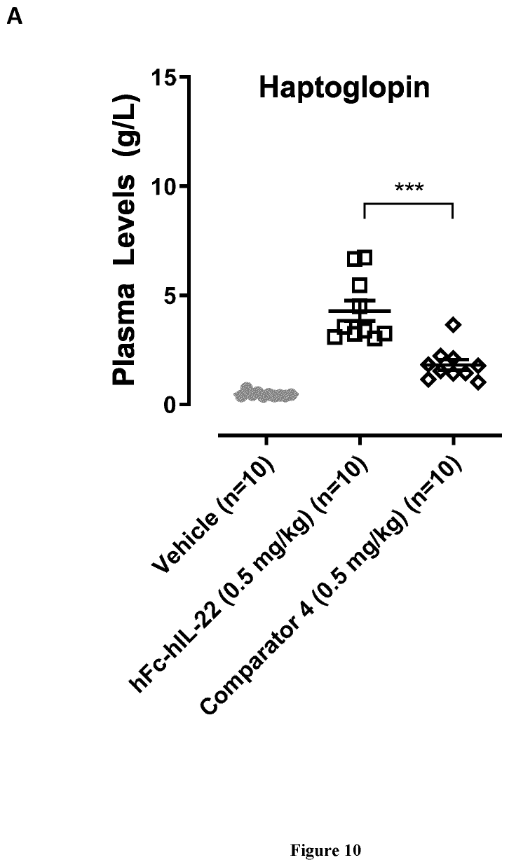

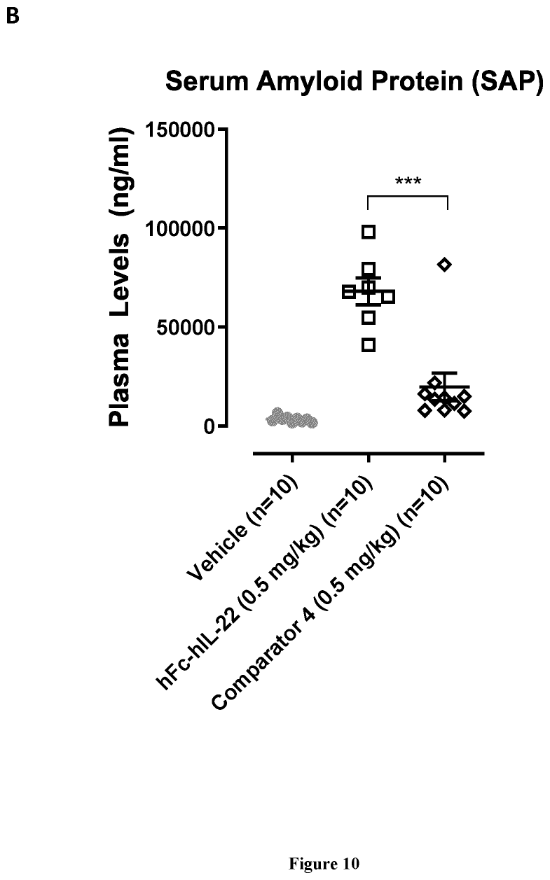

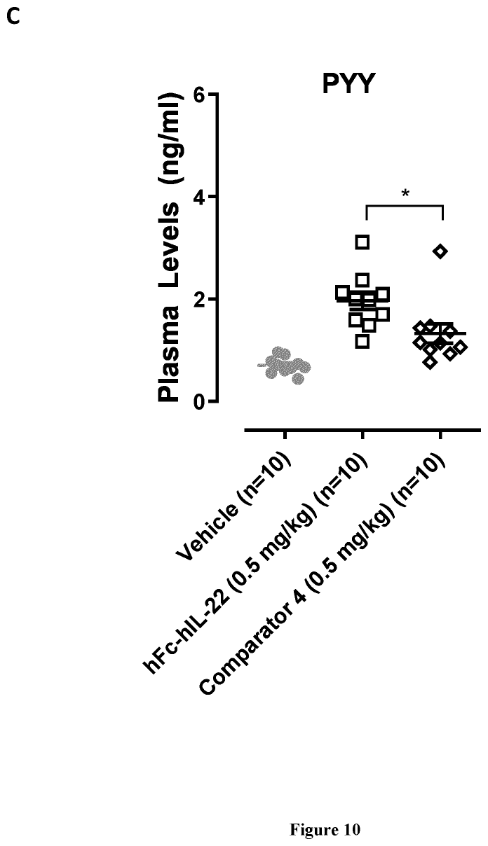

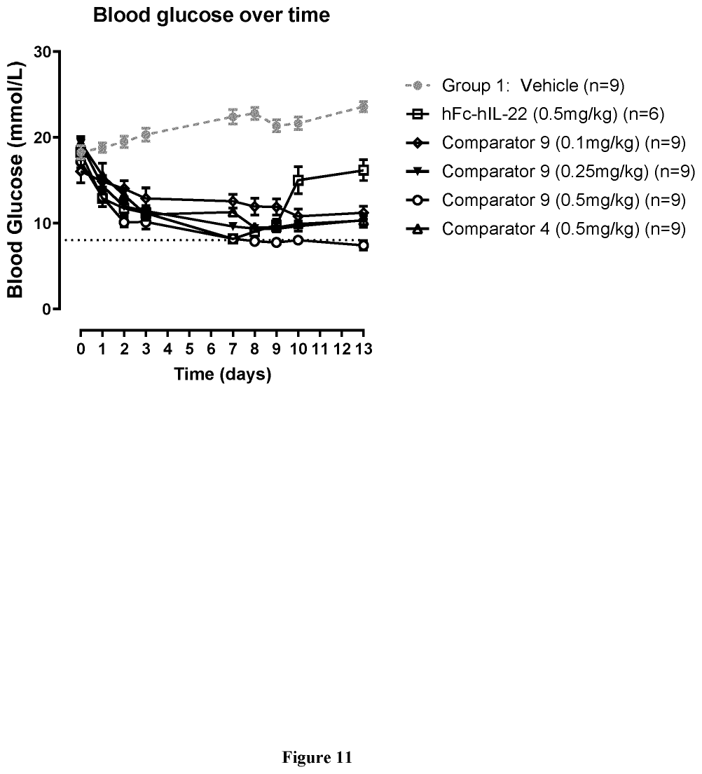

The level of the target engagement biomarkers, haptoglobin, SAP and PYY, as measured at the study end, are shown in FIG. 10A-C, respectively. As can be seen in the graphs, all three target engagement biomarkers were upregulated by Comparator 4 and hFc-hIL-22, moreso by hFc-hIL-22 than Comparator 4.

FIG. 11 shows dose-response data for Comparator 9 (being the same as Comparator 4 but for additional substitutions in two glycosylation sites). All three doses tested (0.1, 0.25 and 0.5 m/kg) were effective at reducing blood glucose over time, and progressively moreso with increasing concentration.

### Conclusion

Both of the tested comparators and hFc-hIL-22 normalised blood glucose in the db/db model, thereby demonstrating an in vivo therapeutic effect. Importantly, no such effect was seen with dosing of hIL-22, demonstrating that the chronic exposure obtained with the long-acting comparators and Fc fusion is necessary for the therapeutic effect. Whilst the mode of action for the anti-diabetic effect is not yet fully elucidated, it is believed that IL-22 effects on the liver (hepatic gluconeogenesis and lipogenesis) are a major contributor.

Food intake was also shown to be reduced by treatment with Comparator 4, thus demonstrating efficacy as an obesity treatment.

Target engagement biomarkers were also observed to be upregulated by the comparators and hFc-hIL-22. The particular biomarkers measured in the db/db mice are known to translate to man.

It is important to note that the circulating half-life (T_{1/2}) of subcutaneously administered hFc-hIL-22 is higher than for Comparator 4, specifically in mice (T_{1/2} of 20 and 8 hours, respectively; see Table 7). Therefore, the exposure of hFc-hIL-22 is higher at steady state. This is further corroborated by the observation that the target engagement biomarkers (haptoglobin, SAP and PYY) were higher in the hFc-hIL-22 group than the Comparator 4 group (FIG. 10) demonstrating higher target engagement per se in the shown experiment. Thus, despite higher exposure and target engagement, the efficacy of the Fc fusion (hFc-hIL-22) was inferior, compared to Comparator 4, in the last three days of a 16-day dosing study. The data hence show that Comparator 4 demonstrates good therapeutic efficacy in a mouse model of diabetes and liver disease. As the particular biomarkers measured in the db/db mice are known to translate to man, it is reasonable to predict that such therapeutic efficacy translates too. Again, similar results can be expected for the derivatives of the invention comprising fatty monoacids instead.

### Example 6—In Vivo Efficacy Study in Liver Injury (i) of Comparators Comprising Fatty Diacids

This study was designed to investigate the effect of dosing with comparators of the derivatives of the invention in a liver injury mouse model. The study was done in preventive mode, meaning that liver injury was only induced after dosing had been initiated.

### Methods

10 week-old C57Bl/6Rj mice were obtained and acclimatised for one week before study start. Liver injury was induced with a single intraperitoneal dose (300 mg/kg, 20 ml/kg) of APAP. Comparators 4 and 9 were dosed at 1.5 mg/kg subcutaneously two hours prior to APAP dosing, alongside vehicle controls (n=5-10). The study was terminated 24 hours after APAP dosing. A terminal bleed was secured for measurement of plasma alanine transaminase (ALT) and aspartate transaminase (AST).

Blood samples were collected in heparinised tubes and plasma was separated and stored at −80° ° C. until analysis. ALT and AST were measured using commercial kits (Roche Diagnostics) on the COBAS c501 autoanalyser according to the manufacturer's instructions.

Livers were subjected to formalin fixation and paraffin embedding for histological analysis.

Proliferation was measured via ki67 immunohistochemistry (IHC) staining. IHC-positive staining was quantified by image analysis using VIS software (Visiopharm, Denmark).

Apoptosis was measured in a terminal deoxynucleotidyl transferase dUTP nick end labeling (TUNEL) assay. In brief, slides with paraffin-embedded sections were de-paraffinated in xylene and rehydrated in series of graded ethanol. The slides were pretreated with proteinase K and endogenous peroxidase activity was blocked with hydrogen peroxide. The TUNEL mixture (In Situ Cell Death Detection Kit, POD, Roche) was added to the slides, followed by amplification with horseradish peroxidase (HRP) and visualization by diaminobenzidine (DAB) (Chromogen). Finally, the slides were counterstained in hematoxylin and cover-slipped.

### Results

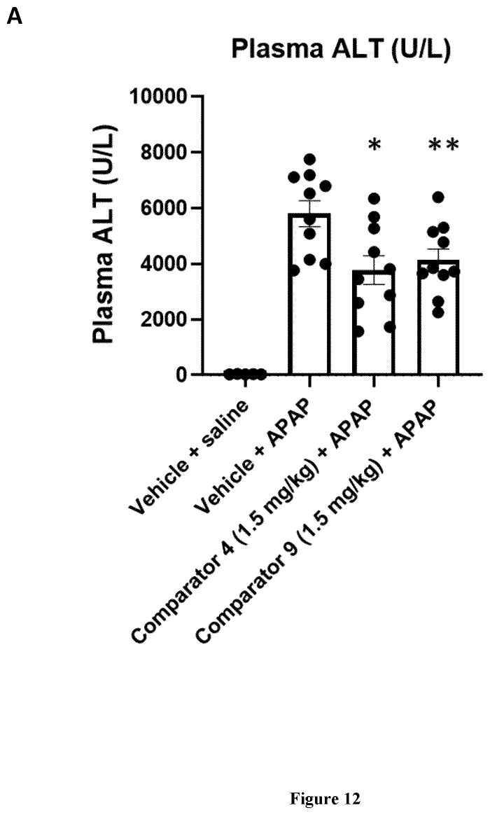

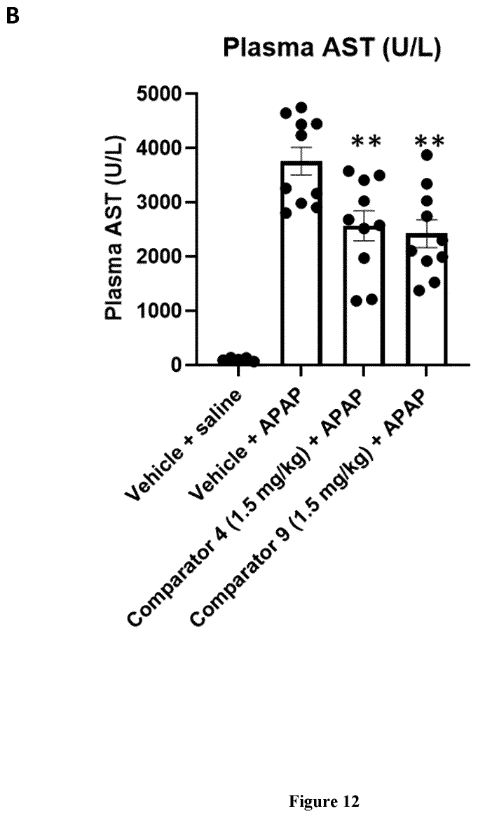

Plasma levels of ALT and AST at the termination of the study are shown in FIGS. 12A and 12B, respectively. The amount of ALT and AST was shown to be significantly reduced in mice treated with Comparator 4 or 9 prior to liver injury compared to the vehicle/APAP control.

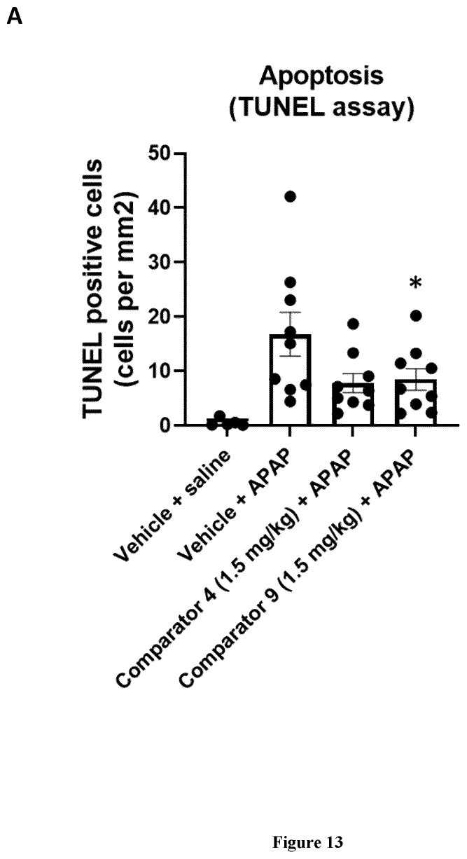

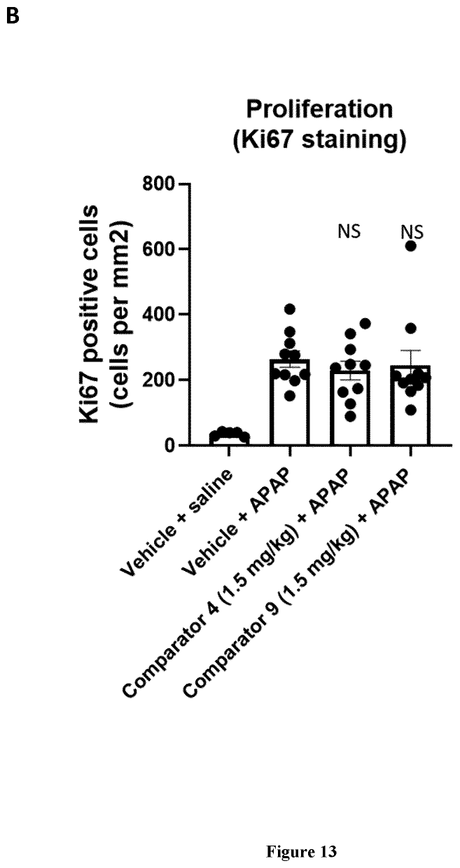

The number of TUNEL- and ki67-positive cells at the termination of the study are shown in FIGS. 13A and 13B, respectively. The amount of TUNEL-positive cells was (significantly) reduced in mice treated with Comparator 4 or 9 prior to liver injury compared to the vehicle/APAP control. The amount of ki67-positive cells was comparable across the APAP-treated groups.

### Conclusion

ALT and AST are liver enzymes used as indicators of liver damage. Comparators 4 and 9 were hence shown to protect the liver against injury induced by APAP. Based on the vast amount of data highlighting similar potencies and half-lives it is reasonable to expect similar effects from other comparators and indeed derivatives disclosed herein based on the vast amount of data highlighting similar potencies and half-lives.

The results of the TUNEL assay showed that Comparators 4 and 9 protected against apoptosis caused by liver injury compared to the vehicle/APAP control. Cellular proliferation, however, was unaffected by these comparators. As proliferation is upregulated physiologically as a response to injury (as seen in control), the results demonstrate the proliferative action of Comparators 4 and 9 as reduced injury is not followed by reduced proliferation (the ratio of proliferation over injury is increased).

The data hence show that the comparators demonstrate good efficacy in protecting against liver injury in a mouse model. The particular biomarkers measured in the mice are known to translate to man, hence it is reasonable to predict that the observed protection would translate too. Again, similar results can be expected for the derivatives of the invention comprising fatty monoacids instead.

### Example 7—In Vivo Efficacy Study in Lung Injury of Comparators Comprising Fatty Diacids

This study was designed to investigate the effect of dosing with a comparator for the derivatives of the invention in a lung injury rat model. The study was done in both preventive and treatment mode, meaning that dosing was initiated before the lung injury was induced and continued thereafter.

### Methods

To induce lung injury, 100 μl of bleomycin was administered to the lungs of male Sprague Dawley rats by oropharyngeal aspiration as a single dose on Day 1 (Groups 2 to 6). Saline was administered as a negative control (Group 1).

Animals in Groups 3, 4 and 5 were dosed (by subcutaneous injection) once daily with Comparator 9 at 0.5, 1.5 or 4.5 mg/kg respectively, from Day −1 to Day 3. Animals in Group 6 were dosed (by oral gavage) once daily with prednisolone at 10 mg/kg from Day −1 to Day 3.

In order to measure soluble collagen in bronchoalveolar lavage fluid (BALF) from the rats, lungs were lavaged (3×4 ml) with sterile PBS (without calcium and magnesium) including added protease inhibitor cocktail, and the lavages per animal placed into one tube. Soluble collagen was measured in BALF supernatant using Soluble Collagen Assay Sircol S1000 (Biocolor) (Charles River Laboratories).

All animals were submitted for necropsy on Day 4 (terminal euthanasia). The right lung was collected for histopathological examination from all animals and inflation-fixed with 10% neutral buffered formalin (NBF) before being immersion-fixed in NBF. Three parallel longitudinal sections were trimmed from the right caudal lung lobe and mounted in cassette 01. The right cranial, middle and accessory lung lobes were also sectioned longitudinally and mounted in cassette 02.

Two slides were made from each cassette; one slide was stained with haematoxylin and eosin (H&E), while the other was stained with haematoxylin and picrosirius red (H&PSR).

Each slide was then assigned a random number using a random number generator. The identification key was recorded in a Microsoft Excel spreadsheet and a copy was provided to the study pathologist following slide evaluation. The six sections per lung were therefore read blind.

A veterinary pathologist then scored each section on each H&E-stained slide for severity of inflammation (where 0=absent, 1=minimal, 2=mild, 3=moderate and 4=severe). The mean and median score per Group was calculated. The pathologist also scored each section on each H&PSR-stained slide for severity of fibrosis (using a modified Ashcroft score from 0=low to 8=high). The mean and median score per Group was calculated and subjected to non-parametric ANOVA, Kruskal-Wallis post-test analysis.

### Results

A summary of the microscopic findings is shown in Table 14, which reveals the mean and median scores for inflammation and fibrosis for each Group.

TABLE 14
Summary of microscopic findings in a lung injury rat model

| | Group | | | | | |
|---|---|---|---|---|---|---|
| | | | 3 Bleo/ | 4 Bleo/ | 5 Bleo/ | |
| | | | Compar- | Compar- | Compar- | |
| | 1 saline/ | 2 Bleo/ | ator 9 | ator 9 | ator 9 | 6 Bleo/ |
| | vehicle | vehicle | 0.5 mg/kg | 1.5 mg/kg | 4.5 mg/kg | Pred |
| Fibrosis | Saline | Bleomycin | | | | |
| induced | | | | | | |
| No. | 10 | 10 | 10 | 10 | 10 | 10 |
| Animals | | | | | | |
| Exam- | | | | | | |
| ined | | | | | | |

Inflammation

| | | | | | | |
|---|---|---|---|---|---|---|
| Group | 0.1 | 1.6 | 1.3 | 1.3 | 1.0 | 1.1 |
| Mean | | | | | | |
| Group | 0.0 | 2.0 | 1.0 | 1.0 | 1.0 | 1.0 |
| Median | | | | | | |

Fibrosis

| | | | | | | |
|---|---|---|---|---|---|---|
| Group | 0.0 | 1.2 | 0.8 | 0.9 | 0.7 | 0.9 |
| Mean | | | | | | |
| Group | 0.0 | 1.0 | 1.0 | 1.0 | 0.0 | 1.0 |
| Median | | | | | | |

As evidenced by comparing Groups 1 and 2 in Table 14, bleomycin induced both lung inflammation and fibrosis in the rat model. The mean and median scores were lower in Groups 3-5, i.e. rats treated with Comparator 9. These lower scores were comparable with those seen in rats treated with prednisolone (Group 6).

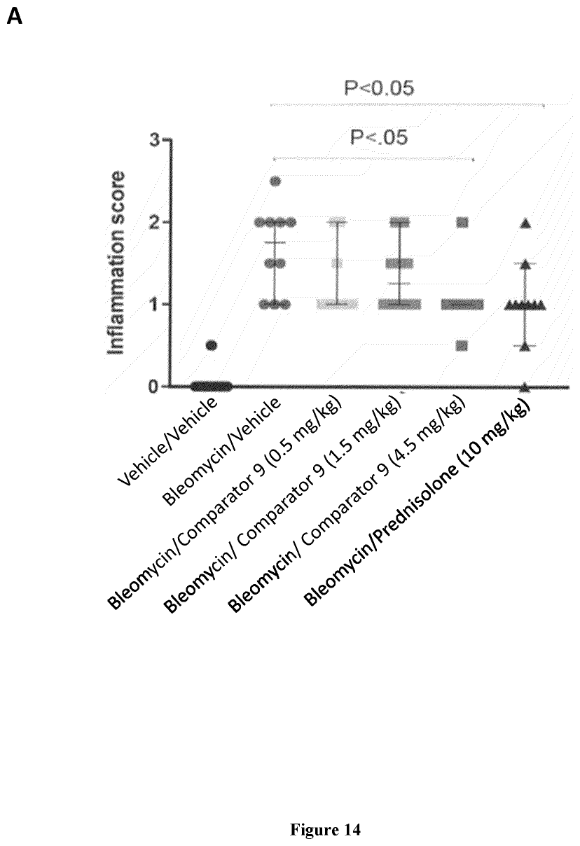

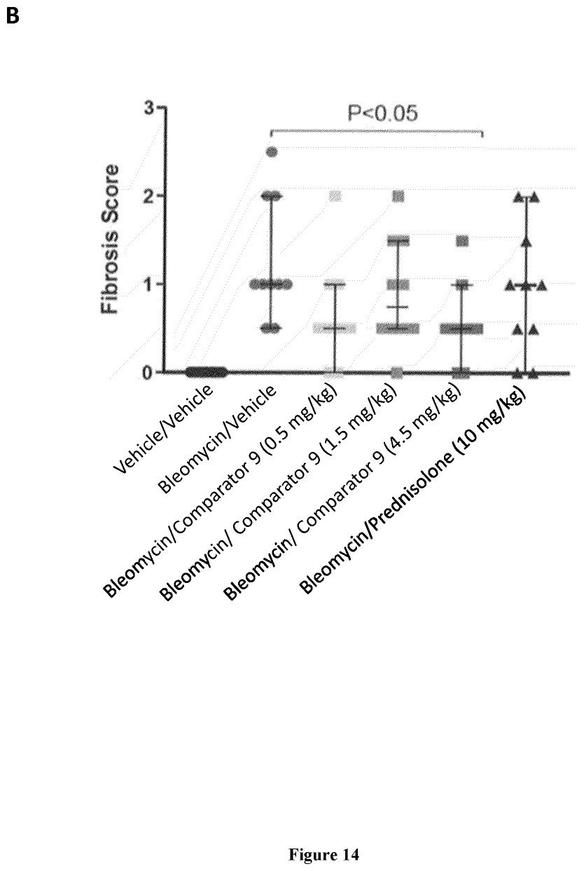

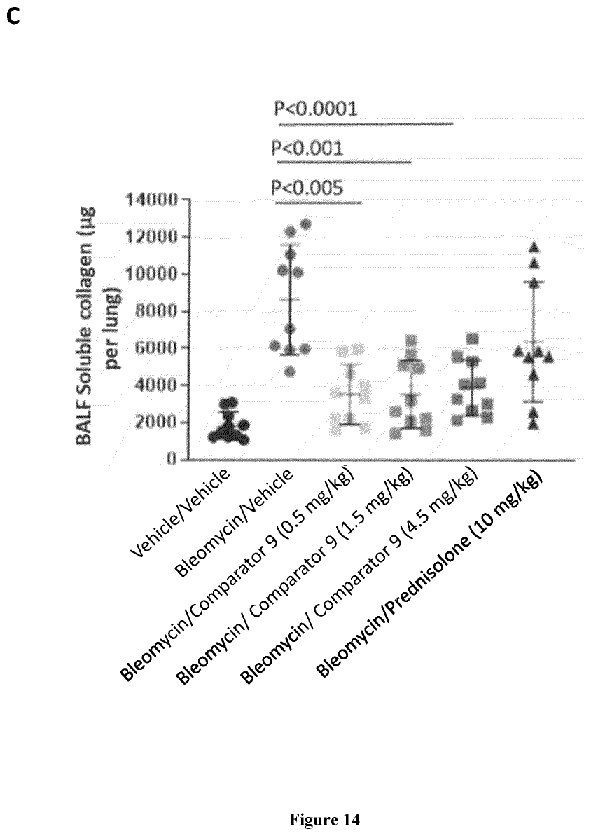

Median inflammation and fibrosis scores for each animal in the study are also shown in FIGS. 14A and 14B, respectively.

As illustrated in FIG. 14A, the Group median inflammation score was increased in the bleomycin/vehicle controls (Group 2) compared with negative controls (Group 1). The Group median inflammation scores were decreased in rats treated with Comparator 9 (and significantly decreased in high dose Group 5) and prednisolone (Group 6) compared with bleomycin/vehicle controls.

As illustrated in FIG. 14B, the Group median fibrosis score was increased in the bleomycin/vehicle controls (Group 2) compared with negative controls (Group 1). However, Group median fibrosis scores were decreased in rats treated with Comparator 9 (and significantly decreased in high dose Group 5) compared with bleomycin/vehicle controls but not with the control prednisolone.

As illustrated in FIG. 14C, the amount of soluble collagen in BALF after bleomycin-induced lung injury was increased in the bleomycin/vehicle controls (Group 2) compared with negative controls (Group 1), and this was not reduced by treatment with prednisolone (Group 6). However, a significantly reduced amount of soluble collagen was observed in BALF from rats treated with Comparator 9 (across all doses tested) compared with bleomycin/vehicle controls. As soluble collagen in BALF is a read-out for fibrosis, these results confirm the histology data reported immediately above.

### Conclusion

The results of the microscopic studies showed that the comparator for derivatives of the invention was able to prevent and/or reduce bleomycin-induced lung inflammation and fibrosis in a rat model. The effects seen with respect to inflammation were comparable to those observed with prednisolone, a corticosteroid known for treating lung inflammation. However, the comparator had a unique action on fibrosis, not seen with prednisolone. Based on the vast amount of data highlighting similar potencies and half-lives it is reasonable to expect similar effects from other comparators and indeed derivatives disclosed herein based on the vast amount of data highlighting similar potencies and half-lives.

### Example 8—In Vivo Efficacy Study in Colitis of Comparators Comprising Fatty Diacids

This study was designed to investigate the effect of dosing with a comparator for the derivatives of the invention in a colitis mouse model. The study was done in both preventive and treatment mode, meaning that dosing was initiated on the same day as the colon inflammation was induced and continued thereafter.

### Methods

Chow-fed female C57Bl/6JRj mice were randomised to five groups (n=8 per group) based on body weight. DSS was used to induce colitis in four of the five groups. These mice received DSS in their drinking water for seven days from study day 0 to 6. In the fifth group, animals received water without DSS and hence served as healthy controls. From study day 0, DSS mice were treated with vehicle, Comparator 9 (at 0.35 mg/kg or 1 mg/kg dosed intraperitoneally) or an IL-22-Fc fusion as comparator (hFc-hIL-22; at 0.5 mg/kg dosed intraperitoneally) once daily for 10 days. Body weight, food and water intake were monitored daily.

On study day 10, blood samples were collected from the mice in EDTA tubes and the plasma was separated and stored at −80° C. until analysis. Regenerating Islet Derived Protein 3 Gamma (Reg3g) was measured in duplicates using an ELISA kit (Cloud-Clone Corp), according to the manufacturer's instructions. Reg3g is a target engagement marker of IL-22.

At termination, the intestines were removed for stereological analysis. Accordingly, the gut was flushed with ice cold saline and its content gently removed before sampling.

The intestine was infiltrated in formalin overnight (Tissue-Tek VIP) and subsequently embedded in blocks of paraffin. The formalin-fixed intestine was then sampled from the proximal to the distal direction using systematic uniform random sampling (SURS) principles, resulting in a total of four slabs and placed in a multi-cassette. All tissue slabs were placed in such a way that identification of individual slabs was possible at a later stage. The paraffin blocks were trimmed and 5 μm top sections were cut and mounted on Superfrost+ object glasses. For the large intestine, another section was cut with a 500 μm distance to the top section, thus giving rise to a total of eight colon sections from each animal.

Colon inflammation volume was measured stereologically, i.e., using a three-dimensional interpretation of two-dimensional cross sections of the colon. The stereological volume estimation was performed using the newCAST system (Visiopharm) on scanned H&E-stained slides. Total gut volume, volume of mucosa, volume of submucosa and muscularis and volume of inflamed tissue were estimated by point counting using a grid system of appropriate size, where all points hitting the structure of interest were counted. The number of points hitting the structure of interest were converted into volume according to the following mathematical relationship:

$$\\ {Vol}_{ref} = \sum p \cdot A ( p ) \cdot t$$

where A(p) is the area per point, p is the total number of points hitting the structure of interest and t is the distance between sections. The mean inflammation volume per group was calculated and subjected to statistical analysis.

Colon morphology was also assessed at termination by viewing the H&E-stained slides.

### Results

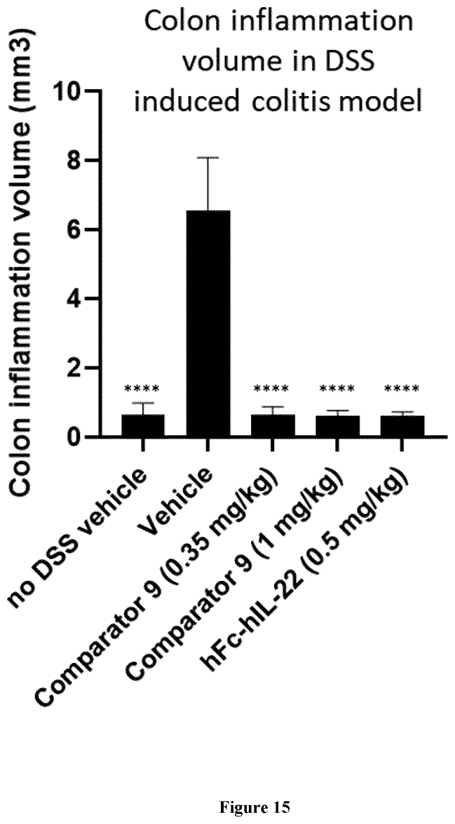

Colon inflammation volume is shown in FIG. 15. Inflammation was shown to be prevented in mice treated with Comparator 9, at either dose, compared to the vehicle control (also containing DSS). Notably, inflammation remained at normal levels in the groups treated with Comparator 9, as evidenced by the colon inflammation volume being the same for the treated groups as the healthy controls (vehicle with no DSS). The same was true for the group treated with hFc-hIL-22.

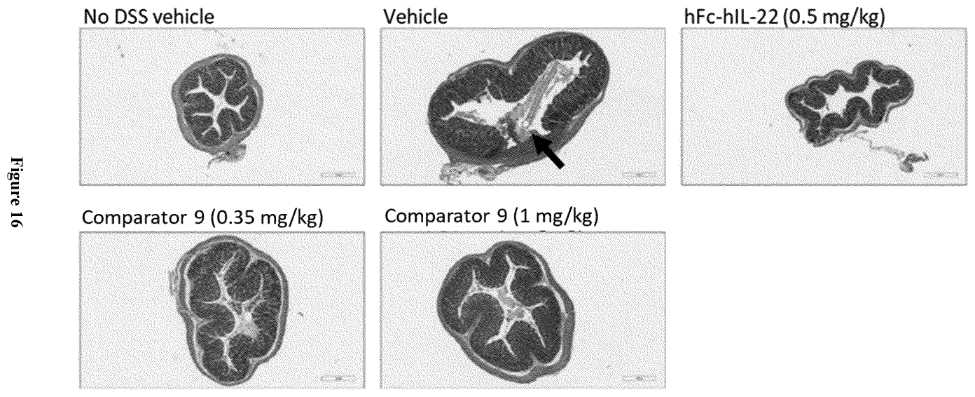

Representative H&E staining images of colon morphology at termination are shown in FIG. 16. Following DSS treatment, mucosal epithelial wounding can be seen in vehicle-treated animals (marked by black arrow), but not in animals treated with Comparator 9 at either dose or hFc-hIL-22. This demonstrates a protective effect on epithelial tissue.

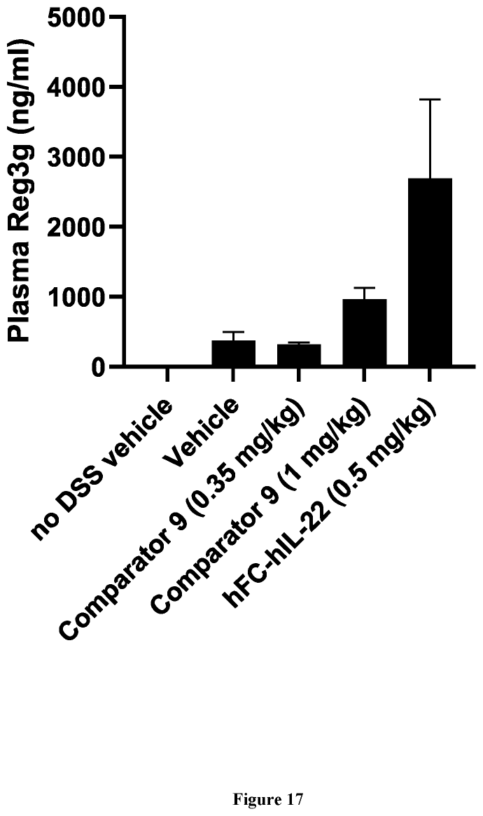

Plasma Reg3g levels are shown in FIG. 17. DSS treatment induced an increase in basal Reg3g levels (compare vehicle to no DSS vehicle). No further increase was detectable in the low dose (0.35 mg/kg) Comparator 9 group, but was seen in the higher dose (1 mg/kg) Comparator 9 group and the hFc-hIL-22 group. The higher Reg3g levels in the hFc-hIL-22 (0.5 mg/kg) group compared to the Comparator 9 (1 mg/kg) group indicated higher target engagement despite the lower dose, which was likely related to the longer half-life in mice of hFc-hIL-22 (T½ of 30 hours for hFc-hIL-22 vs 9.1 hours for Comparator 9).

### Conclusion

The data hence show that a comparator for the derivatives of the invention demonstrates good efficacy in protecting against colitis and mucosal epithelial wounding in a mouse model. This indicates that a new and improved treatment for gut diseases, disorders and conditions has been found. In particular, the findings demonstrate the potential to treat disease characterised by mucosal epithelial damage, such as inflammatory bowel disease. Based on the vast amount of data highlighting similar potencies and half-lives it is reasonable to expect similar effects from other comparators and indeed derivatives disclosed herein based on the vast amount of data highlighting similar potencies and half-lives.

### Example 9—In Vivo Efficacy Study in Liver Injury (ii) of Comparators Comprising Fatty Diacids

This study was designed to investigate the effect of dosing with a comparator for the derivatives of the invention in a second liver injury mouse model (the first being described above in Example 6). The study was done in preventive mode, meaning that liver injury was only induced after dosing had been initiated.

### Methods

C57B16/6j male mice were divided into five groups (n=8 per group). Comparator 4 was dosed at 1 mg/kg intraperitoneally in two of the five groups at −26 hours and −2 hours relative to ConA treatment. Another two groups received vehicle only at these time points. ConA was given to all four groups as an intravenous bolus over a 30-second period at a dose of 15 mg/kg, to induce liver injury. The fifth group received no ConA (vehicle only, as above), as healthy controls.

8 or 24 hours after ConA injection, the mice were placed under isoflurane anaesthesia and the maximal volume of blood was taken by cardiac puncture (using a polypropylene serum gel tube containing a clot activator). Mice receiving no treatment (Group 5) were sacrificed at the 8-hour time point. The blood was mixed with the clotting activation agent in each tube by inverting the tube several times. The tube was maintained for 15 minutes at room temperature and then centrifuged at 2000 g for 10 minutes at 4° C. ALT and AST were measured in the serum samples using an automated system (Konelab 20) according to manufacturer's instructions.

### Results

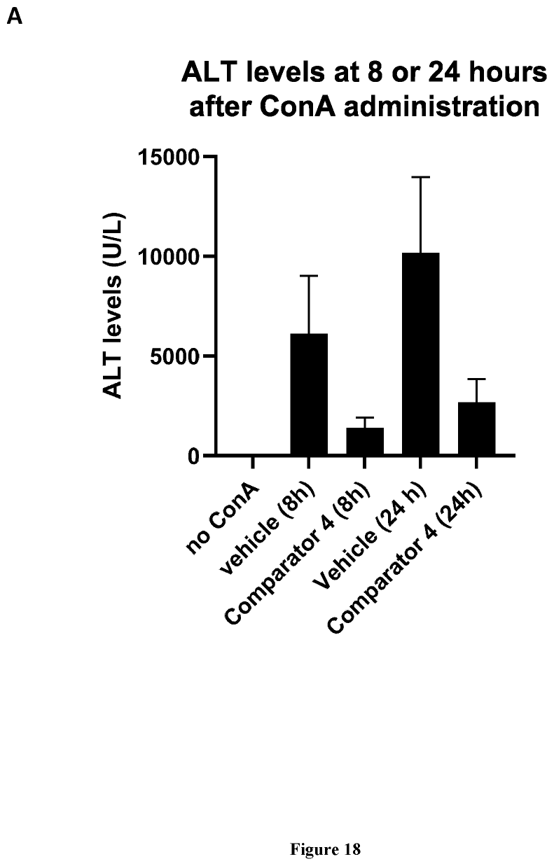

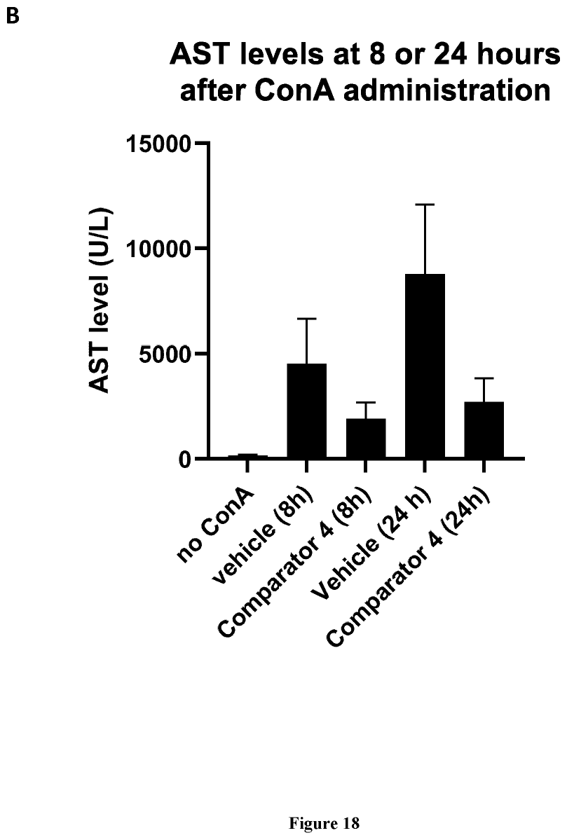

Plasma levels of ALT and AST at the termination of the study are shown in FIGS. 18A and 18B, respectively. The amount of ALT and AST was shown to be reduced in mice treated with Comparator 4 prior to liver injury compared to the vehicle/ConA control, at both time points tested.

### Conclusion

ALT and AST are liver enzymes used as indicators of liver damage. Comparator 4 was hence shown to protect the liver against injury induced by ConA, just as it had against injury induced by APAP in Example 6. The particular biomarkers measured in the mice are known to translate to man, hence it is reasonable to predict that the observed protection would translate too. Based on the vast amount of data highlighting similar potencies and half-lives it is reasonable to expect similar effects from other comparators and indeed derivatives disclosed herein based on the vast amount of data highlighting similar potencies and half-lives.

### Example 10—In Vitro Potency Study of Comparators with R95C or L106C Modifications

### Methods

A reporter gene assay was employed to study potency in BHK cells, which had been triple transfected with IL-22Ra, IL-10Rb and a luciferase with STAT3-induced promoter. This is a highly sensitive, high-throughput assay, which measured IL-22 receptor-mediated STAT3 activation.

A stable reporter BHK cell line was generated using the following plasmids: (i) hIL-10Rb in pcDNA3,1hygro(+), (ii) IL22R in pcDNA3,1(Zeocin) and (iii) 2×KZdel2 in pGL4.20. The cell line hence expressed the human IL-10Rb, human IL-22Ra and luciferase reporter under control of a pSTAT3 driven promoter.

On Day 0 of the assay protocol, the cells were seeded in basal media (for 500 ml: DMEM+Glutamax (Gibco, cat. no.: 31966-021), 10% (w/v) fetal calf serum (FCS; contains albumin) (50 ml) and 1% (w/v) penicillin-streptomycin (P/S) (5 ml)) at 15,000-20,000 cells/well in a 96-well plate (Corning #3842, black, clear bottom). On Day 1, the media was removed by inverting the plate. Fresh basal media was added, at 50 μl per well, and the cells were incubated for 60 minutes.

Comparators 1 and 2 were tested alongside hIL-22 as a further comparator in duplicate.

Thus, 50 μl of a diluted comparator (diluted in basal media) was added to each well and the plate left for four hours. The comparators were therefore 2× diluted, as they were diluted into the 50 μl media already in the wells. The stimulation was ended after four hours by adding 100 μl Steadylite plus reagent (Perkin Elmer cat no. 6066759). The plate was sealed with TopSeal A, shaken at 450 rpm for 15 minutes, then read using Mithras or a similar system no later than after 12 hours.

Data analysis was performed using Graphpad Prism. The half maximal effective concentration (EC₅₀) of each comparator was assessed as a measure of its potency. EC₅₀ was determined using Log(compound) vs response-variable slope (4p). Hill slope was constrained to 1 as standard.

### Results

Table 15 shows the EC₅₀ of the comparators measured in the BHK cell reporter gene assay for IL-22 receptor mediated STAT3 activation.

TABLE 15
EC₅₀ values for key comparators in BHK cell assay

| | | Fold change EC₅₀ |
|---|---|---|
| ID | EC₅₀ (nM) | compared to hIL-22 |
| hIL-22 | 0.07 | NA |
| Comparator 1 | 0.23 | 3 fold |
| Comparator 2 | 0.36 | 5 fold |

As the BHK cell assay contained high amounts of albumin, the measured EC₅₀ incorporated the effect of albumin binding when testing the comparators.

In the BHK cell assay with albumin present, Comparator 1 had a three-fold reduced potency compared to hIL-22 and Comparator 2 had a five-fold reduced potency compared to hIL-22.

### Conclusion

In the BHK cell assay with albumin in the media, Comparators 1 and 2 showed only a three- or five-fold reduction in potency compared to hIL-22. In comparison, and as aforementioned, Genentech reports a 34-fold reduction in in vitro potency for its Fc fusion of IL-22.

Thus, comparators for derivatives of IL-22 are similar in potency with hIL-22 even in the presence of albumin. Cys and Gln substitutions and fatty acid covalent attachment are tolerated in different positions. Indeed, the inventors have shown that conjugation to residues 95 and 106, with the natural amino acids substituted to Cys, has minimal impact on the activity of the IL-22 protein and are therefore considered particularly attractive sites of conjugation. The natural amino acids are R95 and L106. Based on the vast amount of data highlighting similar potencies and half-lives, it is reasonable to expect similar effects from the derivatives of the invention disclosed herein having a fatty monoacid attachment instead of a fatty diacid attachment as exemplified here, albeit the half-lives are expected to be shorter and potency less reduced compared to hIL-22.

### Example 11—In Vitro Potency Study of Comparators with R95C or L106C Modifications

### Methods

These experiments were performed in a different laboratory and therefore not included in Example 10. The assay used was the same, however, the skilled person will appreciate that the equipment and handling of samples may affect the values extracted from the experiments.

A reporter gene assay was employed to study potency in BHK cells, which had been triple transfected with IL-22Ra, IL-10Rb and a luciferase with STAT3-induced promoter. This is a highly sensitive, high-throughput assay, which measured IL-22 receptor-mediated STAT3 activation.

A stable reporter BHK cell line was generated using the following plasmids: (i) hIL-10Rb in pcDNA3,1hygro(+), (ii) IL22R in pcDNA3,1(Zeocin) and (iii) 2×KZdel2 in pGL4.20. The cell line hence expressed the human IL-10Rb, human IL-22Ra and luciferase reporter under control of a pSTAT3 driven promoter.

On Day 0 of the assay protocol, the cells were seeded in basal media (for 500 ml: DMEM+Glutamax (Gibco, cat. no.: 31966-021), 10% (w/v) fetal calf serum (FCS; contains albumin) (50 ml) and 1% (w/v) penicillin-streptomycin (P/S) (5 ml)) at 15,000-20,000 cells/well in a 96-well plate (Corning #3842, black, clear bottom). On Day 1, the media was removed by inverting the plate. Fresh basal media was added, at 50 μl per well, and the cells were incubated for 60 minutes.

Comparators 3 and 19 were tested alongside hIL-22 as a further comparator in duplicate.

Thus, 50 μl of a diluted comparator (diluted in basal media) was added to each well and the plate left for four hours. The comparators were therefore 2× diluted, as they were diluted into the 50 μl media already in the wells. The stimulation was ended after four hours by adding 100 μl Steadylite plus reagent (Perkin Elmer cat no. 6066759). The plate was sealed with TopSeal A, shaken at 450 rpm for 15 minutes, then read using Mithras or a similar system no later than after 12 hours.

Data analysis was performed using Graphpad Prism. The half maximal effective concentration (EC₅₀) of each comparator was assessed as a measure of its potency. EC₅₀ was determined using Log(compound) vs response-variable slope (4p). Hill slope was constrained to 1 as standard.

### Results

Table 16 shows the EC₅₀ of the comparators measured in the BHK cell reporter gene assay for IL-22 receptor mediated STAT3 activation.

TABLE 16
EC₅₀ values for key comparators in BHK cell assay

| | | Fold change EC₅₀ |
|---|---|---|
| ID | EC₅₀ (nM) | compared to hIL-22 |
| hIL-22 | 0.027 | NA |
| Comparator 19 | 0.31 | 11 fold |
| Comparator 3 | 0.085 | 3 fold |

As the BHK cell assay contained high amounts of albumin, the measured EC₅₀ incorporated the effect of albumin binding when testing the comparators.

In the BHK cell assay with albumin present, Comparator 3 had an 11-fold reduced potency compared to hIL-22 and Comparator 19 had a three-fold reduced potency compared to hIL-22.

### Conclusion

In the BHK cell assay with albumin in the media, Comparators 19 and 3 showed only an 11- or three-fold reduction in potency compared to hIL-22. In comparison, and as aforementioned, Genentech reports a 34-fold reduction in in vitro potency for its Fc fusion of IL-22.

### Example 12—In Vitro Potency Study of Derivative 2 with C16 Monoacid Lipidation

### Methods

The method was performed according to the assay described in Example 11 and run in the same experimental setup.

These experiments were performed in a different laboratory and therefore the results were not included in Table 5. The assay, cell line and protocol used were the same, however, the skilled person will appreciate that the equipment and handling of samples may affect the values extracted from the experiments.

Derivative 2 was tested alongside hIL-22 as a comparator in duplicate.

### Results

Table 17 shows the EC₅₀ of the derivative and comparator measured in the BHK cell reporter gene assay for IL-22 receptor mediated STAT3 activation.

TABLE 17
EC₅₀ values for key comparators in BHK cell assay

| | | Fold change EC₅₀ |
|---|---|---|
| ID | EC₅₀ (nM) | compared to hIL-22 |
| hIL-22 | 0.03 | NA |
| Derivative 2 | 0.03 | Equipotent |

As the BHK cell assay contained high amounts of albumin, the measured EC₅₀ incorporated the effect of albumin binding when testing the derivative.

In the BHK cell assay with albumin present, Derivative 2 had no reduced potency compared to hIL-22.

### Conclusion

In the BHK cell assay with albumin in the media, Derivative 2 showed no reduction in potency compared to hIL-22. In comparison, and as aforementioned, Genentech reports a 34-fold reduction in in vitro potency for its Fc fusion of IL-22. This confirms that the claimed derivatives can comprise a G-P-G extension in the N-terminal; however this is not strictly necessary for the biological effect.

While certain features of the invention have been illustrated and described herein, many modifications and equivalents will occur to those of ordinary skill in the art. It is, therefore, to be understood that the claims are intended to cover all such modifications and equivalents as fall within the true spirit of the invention.

1. A derivative of IL-22 comprising a fatty acid covalently attached to an IL-22 protein, wherein the fatty acid is a monoacid.

2. A derivative as claimed in claim 1, wherein the fatty monoacid is covalently attached to the IL-22 protein by a linker.

3. A derivative as claimed in claim 1, wherein the fatty acid is:
(i) of Formula I: 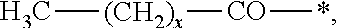
wherein x is an integer in the range of 10-18, optionally 12-18, 14-16 or 16-18, and * designates a point of attachment to the IL-22 protein or linker;
(ii) a C12, C14, C16, C18 or C20 monoacid;
(iii) a C16 or C18 monoacid; and/or
(iv) a C18 monoacid.

4. A derivative as claimed in claim 1, wherein the IL-22 protein is native mature human IL-22 (hIL-22; SEQ ID NO. 1) or a variant thereof.

5. A derivative as claimed in claim 4, wherein the variant:
(i) is a substituted form of hIL-22;
(ii) is substituted at position 1, 21, 35, 64, 113 and/or 114 of hIL-22;
(iii) comprises a substitution of hIL-22 selected from the group consisting of A1C, A1G, A1H, N21C, N21D, N21Q, N35C, N35D, N35H, N35Q, N64C, N64D, N64Q, N64W, Q113C, Q113R, K114C and K114R;
(iv) comprises a Cys residue at position 1 of hIL-22;
(v) comprises a Cys residue at position 95 or 106 of hIL-22;
(vi) comprises a variation within hIL-22 and has at least 10% sequence identity with hIL-22; and/or
(vii) comprises one, two, three, four, five or more variations within hIL-22, wherein said variations are independently selected from the group consisting of deletions, substitutions and insertions.

6. A derivative as claimed in claim 4, wherein the variant:
(i) is an extended form of hIL-22;
(ii) comprises an N-terminal peptide;
(iii) comprises an N-terminal trimer;
(iv) comprises an N-terminal G-P-G; and/or
(v) comprises an N-terminal peptide of up to five, 10, 15, 20, 25, 30, 35, 40, 45 or 50 amino acids.

7. A derivative as claimed in claim 2, wherein the linker comprises:
(i) one or more amino acids, optionally including Glu and/or Lys;
(ii) an oxyethylene glycine unit or multiple linked oxyethylene glycine units, optionally 2-5 such units;
(iii) one or more oligo(ethylene glycol) (OEG) residues;
(iv) an ethylenediamine (C₂DA) group;
(v) an acetamide (Ac) group;
(vi) γGlu-OEG-OEG-C2DA-Ac;
(vii) γGlu-γGlu-γGlu-γGlu-OEG-OEG-εLys-αAc; and/or
(viii) γGlu-OEG-OEG-εLys-αAc.

8. A derivative as claimed in claim 2, wherein the linker is:
(i) a Cys-reactive linker attached to a Cys residue in the hIL-22 or variant thereof;
(ii) attached at position −7, −5, 1, 6, 33, 113, 114 or 153 of the hIL-22 or variant thereof;
(iii) attached to a Cys residue substituted at position 1, 6, 33, 113 or 114 of hIL-22;
(iv) attached to a Cys residue at position −5, −7 or 153 relative to hIL-22;
(v) attached to a Cys residue substituted at position 1 of hIL-22; and/or
(vi) attached to a Cys residue substituted at position 95 or 106 of hIL-22.

9. A derivative as claimed in claim 1, wherein the derivative comprises a C14, C16, C18 or C20 monoacid covalently attached by a linker to a variant of hIL-22, wherein the variant optionally comprises an N-terminal G-P-G and a Cys residue at position 1 of hIL-22 and the linker is optionally attached to said Cys residue.

10. A derivative as claimed in claim 1, wherein the derivative is Derivative 1 or Derivative 2 as identified herein.

11. A pharmaceutical composition comprising a derivative as claimed in claim 1 and a pharmaceutically acceptable vehicle, wherein the pharmaceutical composition is suitable for administration by inhalation, by injection, topically, orally or ocularly, optionally wherein the injection is intraperitoneal, subcutaneous or intravenous.

12. A derivative as claimed in claim 1 or a pharmaceutical composition as claimed in claim 11, for use in therapy.

13. A derivative as claimed in claim 1 or a pharmaceutical composition as claimed in claim 11, for use in a method of therapy, said method comprising administering a daily dose of between 0.001 μg/kg of body weight and 10 mg/kg of body weight of the derivative.

14. A derivative as claimed in claim 1 or a pharmaceutical composition as claimed in claim 11, for use in a method of treating a metabolic, liver, pulmonary, gut, kidney, central nervous system (CNS) or skin disease, disorder or condition.

15. A derivative or pharmaceutical composition as claimed in claim 14, wherein:
(i) the metabolic disease, disorder or condition is obesity, diabetes type 1, diabetes type 2, hyperlipidemia, hyperglycemia or hyperinsulinemia;
(ii) the liver disease, disorder or condition is non-alcoholic fatty liver disease (NAFLD), non-alcoholic steatohepatitis (NASH), cirrhosis, alcoholic hepatitis, acute liver failure, chronic liver failure, acute-on-chronic liver failure (ACLF), acetaminophen induced liver toxicity, acute liver injury, sclerosing cholangitis, biliary cirrhosis or a pathological condition caused by surgery or transplantation;
(iii) the pulmonary disease, disorder or condition is chronic obstructive pulmonary disease (COPD), cystic fibrosis, bronchiectasis, idiopathic pulmonary fibrosis, acute respiratory distress syndrome, a chemical injury, a viral infection, a bacterial infection or a fungal infection;
(iv) the gut disease, disorder or condition is inflammatory bowel disease (IBD), ulcerative colitis, Crohn's disease, graft-versus-host-disease (GvHD), a chemical injury, a viral infection or a bacterial infection;
(v) the kidney disease, disorder or condition is acute kidney disease or chronic kidney disease;
(vi) the CNS disease, disorder or condition is multiple sclerosis; or
(vii) the skin disease, disorder or condition is a wound, inflammatory disease or GvHD.
